SEQUENCE LISTING (2 sequences)

SEQ ID NO: 1 - DNA, 2771 bp, Homo sapiens
tggggctcttgagctataagtggcacctcagggccctgacgggcgtcttgccatgctgctcctgggcctgctgctgctgctgcccctgctggctggcgcccgcctgctgtggaactggtggaagctccggagcctccacctcccgcctcttgccccgggcttcttgcacttgctgcagcccgacctcccaatctatctgcttggcctgactcagaaattcgggcccatctacaggctccaccttgggctgcaaggtgagaggctgatctcgctctggccctcaccataggagggggcggaggtgacggagagggtcctctctccgctgacgctgctttggctgtctcccagatgtggtggtgctgaactccaagaggaccattgaggaagccatggtcaaaaagtgggcagactttgctggcagacctgagccacttacctgtaagggctgggggcattttttctttcttaaaaaaatttttttttaagagatgggttcttgctatgctgcccaggctggtcttaaattcctagtctcaaatgatcctcccacctcagcctcaagtgtgagccacctttggggcatccccaatccaggtccctggaagctcttgggggggcatatctggtggggagaaagcaggggttggggaggccgaagaaggtcaggccctcagctgccttcatcagttcccaccctccagcccccacctcctcctgcagacaagctggtgtctaggaactacccggacctgtccttgggagactactccctgctctggaaagcccacaagaagctcacccgctcagccctgctgctgggcatccgtgactccatggagccagtggtggagcagctgacccaggagttctgtgaggtaaggctgggctcctgaggccacctcgggtcagcctcgcctctcacagtagcccccgccctgcccgctgcacagcggcctgctgaactcacactgtttctccacagcgcatgagagcccagcccggcacccctgtggccattgaggaggaattctctctcctcacctgcagcatcatctgttacctcaccttcggagacaagatcaaggtgcctcacagcccctcaggcccacccccagcccctccctgagcctctccttgtcctgaactgaaagtactccctccttttctggcaggacgacaacttaatgcctgcctattacaaatgtatccaggaggtgttaaaaacctggagccactggtccatccaaattgtggacgtgattccctttctcagggtgaggacctggagcctagacacccctgggttgtaggggagaggctggggtggagggagaggctccttcccacagctgcattctcatgcttcctgccgcagttcttccccaatccaggtctccggaggctgaagcaggccatagagaagagggatcacatcgtggagatgcagctgaggcagcacaaggtggggactgtacgtggacggcctcccctcggcccacagccagtgatgctaccggcctcagcattgctatgaggcgggttcttttgcataccccagttatgggcctgttgccactctgtactcctctccccaggccagccgctcagcccgctcctttcaccctctgcaggagagcctcgtggcaggccagtggagggacatgatggactacatgctccaaggggtggcgcagccgagcatggaagagggctctggacagctcctggaagggcacgtgcacatggctgcagtggacctcctgatcggtggcactgagaccacagcaaacaccctctcctgggccgtggtttttttgcttcaccaccctgaggtgcgtcctggggacaagcaaaaggctccttcccagcaacctggccagggcggtgggcaccctcactcagctctgagcactgtgcggctggggctgtgcttgcctcaccggcactcaggctcactgggttgctgagggagcggctggaggctgggcagctgtgggctgctggggcaggactccacccgatcattccccagattcagcagcgactgcaggaggagctagaccacgaactgggccctggtgcctccagctcccgggtcccctacaaggaccatgcacggctgcccttgctcaatgccaccatcgccgaggtgctgcgcctgcggcccgttgtgcccttagccttgccccaccgcaccacacggcccagcaggtgactcccgagggttggggatgagtgaggaaagcccgagcccagggaggtcctggccagcctctaactccagcccccttcagcatctccggctacgacatccctgagggcacagtcatcattccgaacctccaaggcgcccacctggatgagacggtctgggagaggccacatgagttctggcctggtatgtgggggccgggggcctgccgtcaaaatgtggtggaggctggtccccgctgccgctgaacgcctccccacccacctgtccacccgcccgcagatcgcttcctggagccaggcaagaactccagagctctggccttcggctgcggtgcccgcgtgtgcctgggcgagccgctggcgcgcctggagctcttcgtggtgctgacccgactgctgcaggccttcacgctgctgtcctccggggacgccctgccctccctgcagcccctgccccactgcagtgtcatcctcaagatgcagcctttccaagtgcggctgcagccccgggggatgggggcccacagcccgggccagagccagtgatggggcag

SEQ ID NO: 2 - protein, 494 aa, Homo sapiens [sequence omitted]

(19) United States
(12) Patent Application Publication
(10) Pub. No.: US 2019/0187141 A1
(43) Pub. Date: Jun. 20, 2019

(54) Biomarkers for Detection and Treatment of Mast Cell Activity-Associated Disorders

(71) Applicant: Arete Discoveries, Inc., Weston, MA (US)

(72) Inventor: Jack B. COWIE, III, Weston, MA (US)

(21) Appl. No.: 16/318,847

(22) PCT Filed: Jul. 18, 2017
(86) PCT No.: PCT/US17/42596

§ 371 Date: Jan. 18, 2019

Related U.S. Application Data
(60) Provisional application No. 62/364,103, filed on Jul. 19, 2016.

(51) Int. Cl. G01N 33/564; A61K 45/06; A61P 25/28

(52) U.S. Cl. CPC G01N 33/564; A61K 45/06; A61P 25/28; G01N 2800/60; G01N 2800/52; G01N 2800/54; G01N 2800/285

(57) ABSTRACT

The present invention provides biomarkers for efficient and accurate characterization of mast cell activity-associated diseases, disorders and/or conditions. In particular, the present invention provides biomarkers expressed in mast cell activity-associated diseases, disorders and/or conditions. Those biomarkers, used alone or in combination, may permit more accurate robust characterization of mast cell activity-associated diseases, disorders and/or conditions, resulting in more precise determination of their diagnosis and treatment, and in particular, prediction of the occurrence of MS relapse.

### CROSS REFERENCE TO RELATED APPLICATION

This application claims benefit under 35 U.S.C. 119(e) of U.S. Provisional Application No. 62/364,103 filed Jul. 19, 2016, the contents of which are incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 17, 2017, is named 2012423-0003_.txt and is 8,299 bytes in size.

### BACKGROUND

The pharmaceutical industry has recognized a profound need for identification of biomarkers that correlate with and/or are predictive or prognostic for particular disease events or therapeutic outcomes. Indeed, the Biomarker Qualification Group of the Food and Drug Administration has recently extended its license to the Global Online Biomarker Database (GOBIOM) and now commonly approves therapeutic regimens for administration to populations in which a biomarker has been detected.

The pharmaceutical industry has further recognized that, in many cases, establishment of biomarkers for a particular disease, disorder, or condition, can also define appropriate therapeutic approaches for that disease, disorder and/or condition.

### SUMMARY OF THE INVENTION

The present disclosure encompasses the insight that mast cells and or mast cell metabolites may play important roles in initiation, development, maintenance, and/or relapse of multiple sclerosis (MS) and/or other inflammatory diseases, disorders and/or conditions, and in particular other neuroinflammatory diseases, disorders and/or conditions. Among other things, the present disclosure provides the insight that mast cell metabolites may contribute to enhanced localization of T cells and/or mast cells at central nervous system (CNS) sites. Without wishing to be bound by any particular theory, the present disclosure proposes that mast cell metabolites as described herein may contribute to one or more of increased immune cell (e.g., T cell, mast cells) traversal of the blood brain barrier (BBB) and/or lymphatic vessels, for example by increasing or modulating permeability (at least with respect to immune cells) of the BBB and/or lymphatic vessels, which allows, inter alia, influx of immune cells (e.g., T cells, mast cells) into the CNS, targeting of immune cells (e.g., T cells, mast cells) to one or more particular CNS sites (e.g., to the myelin sheath, to the basal lamina of the BBB), etc.

In some embodiments, the present disclosure defines biological pathways not previously understood to be implicated in initiation, development, maintenance and/or relapse of MS and/or another inflammatory disease, disorder and/or condition.

Among other things, the present disclosure defines one or more useful biomarkers relevant to MS and/or to one or more other demyelinating, autoimmune or inflammatory diseases, disorders and/or conditions. For example, in some embodiments, the present disclosure defines one or more biomarkers of neuroinflammation. The present disclosure identifies the source of a problem in prior work to identify useful biomarkers in that such efforts typically focused on the wrong biological pathways, events, and/or timing. The present disclosure teaches relevance and import of certain events associated with mast cell activity and/or degranulation to MS and/or to one or more other inflammatory diseases, disorders and/or conditions, and provides technologies that define biomarkers for such events.

In some embodiments, the present disclosure provides technologies that identify one or more early biomarkers of development and/or relapse of a neuroinflammatory disease, disorder and/or condition.

In some embodiments, a biomarker as described herein is a mast cell activity (e.g., activation and/or degranulation) marker. In some embodiments, a biomarker as described herein is or comprises level and/or activity of a mast cell metabolite or mast cell activity factor, for example, a cytokine, a preformed granule-associated metabolite, a lipid derived metabolite, chromogranin A, an immunoglobulin (e.g., IgE), a nucleic acid (e.g., RNA or mRNA) and combinations thereof. In some embodiments, a cytokine is IL-1, IL-2, IL-3, IL-4, IL-5, IL-6, IL-8, IL-17, IL-33, TNF-α, TGF-β, GM-CSF, MIP-1α, MIP-1β, INFγ, eosinophilic chemotactic factor or combinations thereof. In some embodiments, a preformed granule-associated meditator is histamine, N-methylhistamine, a proteoglycan, a neutral protease or combinations thereof. In some embodiments, a proteoglycan is heparin and/or chondroitin sulphate. In some embodiments, a neutral protease is tryptase, chymase, carboxypeptidase and/or cathepsin G. In some embodiments, a lipid derived metabolite is a prostaglandin, a thromboxane and/or a leukotriene. In some embodiments, a leukotriene is leukotriene E₄, leukotriene B₄ and/or leukotriene C₄. In some embodiments, a prostaglandin includes, but is not limited to, prostaglandin D₂, prostaglandin E₂, 11β-PGF_{2α}, tetranor-PGDM and/or other metabolites thereof.

In one aspect, the present disclosure provides methods of treating a mast cell activity-associated disorder in a subject in need of treatment comprising administering a mast cell activity inhibitor to a subject, wherein presence of a mast cell activity biomarker has been detected in a sample from the subject. In some embodiments, the subject is diagnosed as suffering from, or susceptible to, the mast cell activity-associated disorder. In some embodiments, the mast cell activity inhibitor is or comprises a T-cell receptor ligand that competes with or inhibits one or more T-cell activating factors, a mast cell adhesion inhibitor, an inhibitor of mast cell degranulation products, a B-cell activity inhibitor, an inhibitor of transgranulation and/or a gene therapy agent that corrects a mutation in the cytochrome P450 family. In some embodiments, a sample is selected from the group consisting of whole blood, plasma, serum, urine, cerebrospinal fluid and lymphatic fluid.

In one aspect, the present disclosure provides methods of treating a mast cell activity-associated disorder in a subject in need of treatment, comprising administering one or both of a first agent selected from the group consisting of chondroitin, methylsulfonylmethane (MSM), glucosamine, an H₁ receptor antagonist, an H₂ receptor antagonist, and combinations thereof; and a second agent selected from the group consisting of selective serotonin reuptake inhibitors (SSRIs), norepinephrine-dopamine reuptake inhibitors (NDRIs), and combinations thereof, to the subject if presence of a mast cell activity biomarker has been detected in a sample from the subject, so that the subject is treated with the first and second agents in combination. In some embodiments, the first agent and second agent are administered at the same time or are administered sequentially.

In one aspect, the present disclosure provides methods of treating a mast cell activity-associated disorder in a subject in need of treatment, comprising administering to a subject a nanobot agent adapted to detect presence of a mast cell activity biomarker and to deliver a mast cell activity inhibitor, so that the mast cell activity inhibitor is administered when the biomarker is detected.

In one aspect, the present disclosure provides methods of diagnosing a subject as susceptible to MS relapse comprising detecting a presence, level and/or location of a mast cell activity biomarker in the subject or in a sample from the subject; predicting the occurrence of MS relapse based on the mast cell activity biomarker that is detected; and administering a mast cell activity inhibitor to treat the MS relapse. In some embodiments, a step of detecting comprises contacting the subject or sample with a molecular contrast agent to visualize mast cell degranulation. In some embodiments, a sample is whole blood, plasma, serum, urine, cerebrospinal fluid or lymphatic fluid.

In one aspect, the present disclosure provides methods of determining a mast cell activity biomarker of a mast cell activity-associated disorder comprising determining presence, level and/or location of one or more mast cell activity factors in samples of subjects suffering from or susceptible to the mast cell activity-associated disorder; detecting a correlation between a determined presence, level and/or location of the one or more mast cell activity factors with incidence, severity, or therapeutic response of the mast cell activity-associated disorder, thereby establishing the determined presence, level and/or location as a mast cell activity biomarker for the incidence, severity, or therapeutic response. In some embodiments, correlated determined presence, level and/or location includes a plurality of data points, each representing presence, level and/or location of a different mast cell activity factor. In some embodiments, at least one data point represents a level relative to an established threshold for a particular mast cell activity factor. In some embodiments, incidence, severity, or therapeutic response of the mast cell activity-associated disorder correlates with presence, level and/or location of mast cell activity, mast cell proliferation, mast cell migration, release of cytokines, release of lipid derived metabolites, release of granule-associated metabolites, hydration, inflammation and combinations thereof.

In some embodiments, a mast cell activity-associated disorder is MS. In some embodiments, incidence, severity, or therapeutic response of MS correlates with presence, level and/or location of mast cell activity, mast cell proliferation, mast cell migration, release of cytokines, release of lipid derived metabolites, release of granule-associated metabolites, hydration, inflammation and combinations thereof. In some embodiments, the severity of the mast cell activity-associated disorder correlates with the presence and/or level of prostaglandin D₂ and or histamine.

In some embodiments, a step of determining comprises visualizing T-cell activation factors, B-cell activation factors and/or mast cell degranulation factors in tissue of a mouse model.

In one aspect, the present disclosure provides, methods of detecting a mast cell activity biomarker of a mast cell activity-associated disorder, comprising obtaining a sample from a human patient and detecting in the sample presence, level and/or location of one or more mast cell activity factors determined to be a mast cell activity biomarker of the mast cell activity-associated disorder. In one embodiment, the method comprises a step of comparing the presence, level and/or location of one or more mast cell activity factors with a reference presence, level and/or location of one or more mast cell activity factors. In one embodiment, a sample is selected from the group consisting of whole blood, plasma, serum, urine, cerebrospinal fluid and lymphatic fluid.

Some embodiments of the present invention provide methods of identifying genetic markers (e.g., allelic variant) that predispose an individual to the development of a mast cell activity-associated disorder (e.g., autoimmune disorder, MS). Some embodiments of the invention provide methods of treatment comprising modification of the genetic marker (e.g., allelic variant) to prevent or treat a mast cell activity-associated disorder (e.g., autoimmune disorder, MS).

In one aspect, the present disclosure provides methods of identifying an agent useful in the treatment of a mast cell activity-associated disorder or MS comprising a candidate agent and assessing the agent for a presence, level and/or location of an activity such as stabilizing mast cell activity, inhibiting mast cell proliferation, inhibiting mast cell migration, inhibiting release of cytokines, inhibiting release of lipid derived metabolites, inhibiting release of granule-associated metabolites, enhancing hydration, and/or reducing inflammation.

In one aspect, the present disclosure provides kits for determining a MS susceptibility biomarker in a subject comprising agents for determining a presence, level and/or location of one or more particular mast cell activity factors, which one or more particular mast cell activity factors have been determined to contribute to a mast cell activity biomarker for MS susceptibility.

### BRIEF DESCRIPTION OF THE DRAWINGS

FIG. 1 depicts exemplary data showing mean clinical EAE score at 9-13 days post immunization.

FIG. 2 depicts exemplary data showing body weight change, as a percentage of initial body weight, from day 0 to day 12 post immunization.

FIGS. 3A and 3B depict exemplary data showing level of histamine/N-methylhistamine in urine and serum of naïve and EAE mice.

FIGS. 4A and 4B depict exemplary data showing level of PGDM in urine and serum of naïve and EAE mice.

FIG. 5 depicts exemplary data showing level of histamine/N-methylhistamine in urine and serum of naïve mice and mice with EAE induced by adoptive cell transfer.

FIG. 6 depicts exemplary data showing level of PGDM in urine and serum of naïve mice and mice with EAE induced by adoptive cell transfer.

### DEFINITIONS

Activating agent: As used herein, the term “activating agent” refers to an agent whose presence or level correlates with elevated level or activity of a target, as compared with that observed absent the agent (or with the agent at a different level). In some embodiments, an activating agent is one whose presence or level correlates with a target level or activity that is comparable to or greater than a particular reference level or activity (e.g., that observed under appropriate reference conditions, such as presence of a known activating agent, e.g., a positive control).

Administration: As used herein, the term “administration” refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: As used herein, the term “agent” refers to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amelioration: As used herein, the term “amelioration” refers to the prevention, reduction or palliation of a state, or improvement of the state of a subject. Amelioration includes, but does not require complete recovery or complete prevention of a disease, disorder and/or condition (e.g., a mast cell activity-associated disorder).

Antagonist: As used herein, the term “antagonist” refers to an agent condition, or event whose presence, level, degree, type, or form correlates with decreased level or activity of another agent (i.e., the inhibited agent, or target). In general, an antagonist may be or include an agent of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target); in some embodiments, an antagonist may be indirect (in which case it exerts its influence by other than binding to its target; e.g., by interacting with a regulator of the target, so that level or activity of the target is altered).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-Bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Antigen presenting cell: As used herein, the term “antigen presenting cell” or “APC” refers to cells which process and present antigens to T-cells. Exemplary antigen cells include dendritic cells, macrophages, mast cells and certain activated epithelial cells.

Approximately: As used herein, the term “approximately” or “about,” may be applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the term “associated with” refers to two or more events or entities if the presence, level and/or form of one is correlated with that of the others. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding agent: As used herein, the term “binding agent” refers to any entity that binds to a target of interest as described herein. In many embodiments, a binding agent of interest is one that binds specifically with its target in that it discriminates its target from other potential binding partners in a particular interaction context. In general, a binding agent may be or comprise an entity of any chemical class (e.g., polymer, non-polymer, small molecule, polypeptide, carbohydrate, lipid, nucleic acid, etc). In some embodiments, a binding agent is a single chemical entity. In some embodiments, a binding agent is a complex of two or more discrete chemical entities associated with one another under relevant conditions by non-covalent interactions. For example, those skilled in the art will appreciate that in some embodiments, a binding agent may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple binding agents through linkage of different specific binding moieties with the same generic binding moiety partner. In some embodiments, binding agents are or comprise polypeptides (including, e.g., antibodies or antibody fragments). In some embodiments, binding agents are or comprise small molecules. In some embodiments, binding agents are or comprise nucleic acids. In some embodiments, binding agents are aptamers. In some embodiments, binding agents are polymers; in some embodiments, binding agents are not polymers. In some embodiments, binding agents are non-polymeric in that they lack polymeric moieties. In some embodiments, binding agents are or comprise carbohydrates. In some embodiments, binding agents are or comprise lectins. In some embodiments, binding agents are or comprise peptidomimetics. In some embodiments, binding agents are or comprise scaffold proteins. In some embodiments, binding agents are or comprise mimeotopes. In some embodiments, binding agents are or comprise stapled peptides. In certain embodiments, binding agents are or comprise nucleic acids, such as DNA or RNA.

Biologically active: As used herein, the term “biologically active” refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Biomarker: As used herein, the term “biomarker” refers to a to an entity whose presence, level, or form, correlates with a particular biological event or state of interest, so that it is considered to be a “marker” of that event or state. To give but a few examples, in some embodiments, a biomarker may be or comprise a marker for a particular disease state, or for likelihood that a particular disease, disorder and/or condition may develop, occur, or reoccur. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, and a biomarker is diagnostic, of the relevant biological event or state of interest. A biomarker may be an entity of any chemical class. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is a cell surface marker. In some embodiments, a biomarker is intracellular. In some embodiments, a biomarker is found outside of cells (e.g., is secreted or is otherwise generated or present outside of cells, e.g., in a body fluid such as blood, urine, tears, saliva, cerebrospinal fluid, etc.)

Cancer: As used herein, the terms “cancer”, “malignancy”, “neoplasm”, “tumor”, and “carcinoma” refers to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Carrier: As used herein, “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agents or modalities to a subject receiving the other agents or modalities in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Detection entity: As used herein, the term “detection entity” refers to any element, molecule, functional group, compound, fragment or moiety that is detectable. In some embodiments, a detection entity is provided or utilized alone. In some embodiments, a detection entity is provided and/or utilized in association with (e.g., joined to) another agent. Examples of detection entities include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Diagnostic information: As used herein, the terms “diagnostic information” or “information for use in diagnosis” refer to information that is useful in determining whether a patient has a disease, disorder and/or condition and/or in classifying a disease, disorder and/or condition into a phenotypic category or any category having significance with regard to prognosis of a disease, disorder and/or condition, or likely response to treatment (either treatment in general or any particular treatment) of a disease, disorder and/or condition. Similarly, “diagnosis” refers to providing any type of diagnostic information, including, but not limited to, whether a subject is likely to have or develop a disease, disorder and/or condition, state, staging or characteristic of a disease, disorder and/or condition as manifested in the subject, information related to the nature or classification of a tumor, information related to prognosis and/or information useful in selecting an appropriate treatment. Selection of treatment may include the choice of a particular therapeutic agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc.

Dosage form, unit dosage form or unit dose: As used herein, the terms “dosage form”, “unit dosage form” or “unit dose” refer to refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: As used herein, the term “excipient” refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” refers to a nucleic acid sequence refers to one or more of the following events: (1) production of an RNA template (e.g., a messenger RNA) from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Event: As used herein, the term “event” refers to mast cell activities. In some embodiments, an event may be reflected in localization or migration of mast cells within a tissue or organ. In some embodiments, an event may be reflected in degree or type of interaction with other cell types. In some embodiments, interaction may be transgranulation. In some embodiments, an event may be reflected in degree or type of interaction with one or more particular proteins or mast cell sites.

Gene: As used herein, the term “gene” refers to a DNA sequence in a chromosome that codes for a product (e.g., an RNA product such as a messenger RNA, and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product: As used herein, the term “gene product” or “expression product” refers to an RNA transcribed from the gene (pre- and/or post-processing) or a polypeptide (pre- and/or post-modification) encoded by an RNA transcribed from the gene.

Improve, increase or reduce: As used herein, the terms “improve”, “increase” or “reduce” or grammatical equivalents thereof, refers to values that are relative to a baseline measurement, such as a measurement in the same individual prior to initiation of a treatment described herein, or a measurement in a control individual (or multiple control individuals) in the absence of the treatment described herein. In some embodiments, a “control individual” is an individual afflicted with the same form of disease or injury as an individual being treated.

Inhibitor: As used herein, the term “inhibitor” refers to an agent, condition, or event whose presence, level, degree, type, or form correlates with decreased level or activity of another agent (i.e., the inhibited agent, or target). In general, an inhibitor may be or include an agent of any chemical class including, for example, small molecules, polypeptides, nucleic acids, carbohydrates, lipids, metals, and/or any other entity, condition or event that shows the relevant inhibitory activity. In some embodiments, an inhibitor may be direct (in which case it exerts its influence directly upon its target, for example by binding to the target); in some embodiments, an inhibitor may be indirect (in which case it exerts its influence by interacting with and/or otherwise altering a regulator of the target, so that level and/or activity of the target is reduced).

Kit: As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contain a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

Mast cell activity factor: As used herein, the term “mast cell activity factor” refers to agents whose presence, level, or location correlates with mast cell activity. In some embodiments, a mast cell activity factor is produced by a mast cell (e.g., may be a mast cell metabolite). In some such embodiments, a mast cell activity factor may be a mast cell metabolite whose location (e.g., within granules vs. released) correlates with mast cell activity; alternatively or additionally, in some such embodiments, a mast cell activity factor may be a mast cell metabolite whose production is triggered or increased in association with mast cell activity. In some embodiments, a mast cell activity factor is not produced by mast cells, but is an agent whose presence, level, or location correlates with mast cell activity. Those of ordinary skill in the art will be familiar with various agents that are mast cell activity factors; representative such agents are included in Table 1 herein.

Mast cell metabolite: As used herein, the term “mast cell metabolite” refers to a chemical compound that is produced, and typically released, by mast cells. In some embodiments, a mast cell metabolite is stored in granules in mast cells and is released by degranulation. Alternatively or additionally in some embodiments, a mast cell metabolite is synthesized upon mast cell activation. Those of ordinary skill in the art will be familiar with various agents that are mast cell metabolites; representative such agents are included in Table 1 herein.

Mast cell site: As used herein, the term “mast cell site” refers to a site within a body where mast cells are or recently have been present. In some embodiments, a mast cell site is bone marrow, peripheral blood, connective tissue, CNS (e.g., brain, spinal cord, meninges), mucosa, lymphatic vessels.

Modulator: As used herein, the term “modulator” refers to an entity whose presence or level in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an antagonist or inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder and/or condition. In some embodiments, a patient has been diagnosed with one or more disorders or conditions. In some embodiments, one or more disorders or conditions is or includes cancer, or presence of one or more tumors. In some embodiments, a patient is receiving or has received certain therapy to diagnose and/or to treat a disease, disorder, or condition. In some embodiments, a patient is a subject.

Pharmaceutical composition: As used herein, the term “pharmaceutical composition” refers to a composition in which an active agent is formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” when applied to the carrier, diluent, or excipient used to formulate a composition as disclosed herein means that the carrier, diluent, or excipient must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Physiological condition: As used herein, the term “physiological condition” refers to conditions under which cells or organisms live and/or reproduce. In some embodiments, the term refers to conditions of the external or internal milieu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

Prevention: As used herein, the term “prevention” refers to a delay of onset, and/or reduction in frequency and/or severity of one or more symptoms of a particular disease, disorder and/or condition. In some embodiments, prevention is assessed on a population basis such that an agent is considered to “prevent” a particular disease, disorder and/or condition if a statistically significant decrease in the development, frequency, and/or intensity of one or more symptoms of the disease, disorder and/or condition is observed in a population susceptible to the disease, disorder, and/or condition. Prevention may be considered complete when onset of a disease, disorder and/or condition has been delayed for a predefined period of time.

Reference: As used herein, the term “reference” refers to a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” refers to a biological sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; plasma; serum; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Subject: As used herein, the term “subject” refers to an organism, typically a mammal (e.g., a human, in some embodiments, including prenatal human forms). In some embodiments, a subject is suffering from a relevant disease, disorder and/or condition. In some embodiments, a subject is susceptible to a disease, disorder, or condition. In some embodiments, a subject displays one or more symptoms or characteristics of a disease, disorder and/or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Susceptible to: As used herein, an individual who is “susceptible to” a disease, disorder, or condition (e.g., a mast cell activity-associated disorder) is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from the disease, disorder, or condition).

Symptoms are reduced: As used herein, the term “symptoms are reduced” refers to when one or more symptoms of a particular disease, disorder and/or condition is reduced in magnitude (e.g., intensity, severity, etc.) and/or frequency. For purposes of clarity, a delay in the onset of a particular symptom is considered one form of reducing the frequency of that symptom.

Therapeutically effective amount: As used herein, the term “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder and/or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION OF THE INVENTION

### Mast Cell Activity-Associated Disorders

The present invention encompasses the insight that mast cell activity is implicated in the pathology of many diseases. The present disclosure thus defines mast cell activity-associated diseases, disorders and/or conditions as disorders in which mast cell activity plays a role in its pathology (e.g., signs, symptoms, onset, severity, progression, recurrence). In some embodiments, mast cell activity-associated disorder is a mast cell activation disorder. In some embodiments, mast cell activity may include degranulation. Degranulation is a cellular process that releases molecules (e.g., metabolites) from secretory vesicles (e.g., granules) found inside a cell.

Among other things, the present disclosure teaches that mast cell activity and/or degranulation can trigger and/or promote undesirable and/or damaging events, and furthermore specifically documents that certain aspects of mast cell activity and/or degranulation can be associated with and/or responsible for features of certain inflammatory diseases, disorders and/or conditions, as described herein. Among other things, the present disclosure documents that certain aspects of mast cell activity and/or degranulation can be associated with and/or responsible for features of MS, e.g., susceptibility to MS relapse, MS severity and/or for features of one or more other inflammatory diseases, disorders and/or conditions (e.g., in particular one or more neuroinflammatory diseases, disorders and/or conditions). The present disclosure therefore describes “undesirable” mast cell activity and/or degranulation, and defines certain characteristics and/or markers thereof (i.e., defines mast cell activity biomarkers).

In some embodiments, mast cell activity may be reflected in a change in level and/or location of a mast cell activity factor. In some embodiments, a change in level may involve one or both of increased synthesis and decreased degradation. In some embodiments, a change in location may involve release from a cell (e.g., release of a mast cell metabolite from a mast cell, for example by degranulation). In some embodiments, a change in location may involve increased presence (e.g., of a mast cell activity factor, for example released by a mast cell or by a cell other than a mast cell, in a mast cell site).

In some embodiments, determination of mast cell activity may involve detecting level of a mast cell activity factor gene product (i.e., a product of a gene that encodes a mast cell activity factor). In some embodiments, a mast cell activity factor gene product is or comprises RNA; in some embodiments, a mast cell activity factor gene product is or comprises a polypeptide.

In some embodiments, level of a mast cell activity factor gene product is in a biological sample. In some embodiments, a biological sample is whole blood, plasma, serum, urine, cerebrospinal fluid (CSF) and lymphatic fluid.

In some embodiments, detecting a level of a mast cell activity factor gene product involves determining whether the mast cell activity factor gene product is or is not detected (i.e., detecting presence of the gene product). In some embodiments, detecting a level of a mast cell activity factor gene product involves measuring (e.g., quantifying) a level of the product.

In some embodiments, mast cell activity may be reflected in localization or migration of mast cells within a tissue or organ, for example, in the CNS. In some embodiments, detecting localization or migration of mast cells may involve techniques known in the art such as in vitro and in vivo imaging techniques and immunohistochemistry.

In some embodiments, mast cell activity may be reflected in degree or type of interaction with other cell types, for example, T cells, B cell, macrophages, dendritic cells, lymphocytes, neutrophils, monocytes, basophils, eosinophils, neurons, astrocytes, microglial cells, pericytes and/or endothelial cells of the blood brain barrier. In some embodiments, such interaction may be or comprise transgranulation. In some embodiments, transgranulation includes, for example, binding and/or association of a mast cell with a non-mast cell and/or release of one or more mast cell metabolites directly into the non-mast cell. In some embodiments, a non-mast cell is a T cell or a neuron. In some embodiments, detecting mast cell interaction with other cell types may involve in vitro and in vivo imaging techniques and immunohistochemistry.

In some embodiments, mast cell activity be reflected in degree or type of interaction with one or more particular proteins or sites; in some embodiments, such proteins or sites may be or comprise, for example, myelin or the basal lamina of the BBB. In some embodiments, detecting mast cell interaction with one or more proteins or sites may involve in vitro and in vivo imaging techniques and immunohistochemistry.

Cancer

Among other things, the present disclosure teaches that mast cell activity may be associated with and/or characteristic of one or more features of cancer. Thus, in accordance with some embodiments of the present disclosure, cancer may be considered to be a mast cell activity-associated disorder.

In some embodiments, a cancer that may be a mast cell activity-associated disorder in accordance with the present disclosure may be or comprise bladder cancer, breast cancer, carcinoid, colon cancer, rectal cancer, glioblastoma, liver cancer, lung cancer, non-small cell lung cancer, chronic lymphocytic leukemia, Hodgkins' lymphoma, non-Hodgkin's lymphoma, malignant melanoma, multiple myeloma, neuroblastoma, ovarian cancer, pancreatic cancer, prostate cancer, renal cell carcinoma, throat cancer and uterine cancer.

In some embodiments, the present disclosure teaches that mast cell activity and/or degranulation play a role in the pathology (e.g., signs, symptoms, onset, severity, progression, recurrence) of cancer. Mast cells may play a role in oncological diseases, through mediation of vasculature growth, permeability and metastases (Ammendola et al., Organ der Deutschen Gesellschaft fur Transfusionsmedizin and Immunhamatologie (2016) 43:109-113; Conti et al., Ann. Clin. Lab. Sci. (2007) 37:315-322).

In some embodiments, the present disclosure teaches that a mast cell biomarker as described herein may be a useful biomarker for cancer. For example, in some embodiments and in accordance with the present disclosure, such a mast cell biomarker that may be used as an indicator for cancer, risk of developing cancer, response to a therapeutic intervention, likelihood of cancer progression and/or severity of cancer is determined and/or detected.

Inflammatory Disorders

The present disclosure provides that mast cell activity may be associated with and/or characteristic of one or more features of inflammatory diseases, disorders and/or conditions. Thus, in accordance with some embodiments of the present disclosure, inflammatory disorders may be considered to be mast cell activity-associated disorders.

In some embodiments, an inflammatory disorder that may be a mast cell activity-associated disorder in accordance with the present disclosure may be or comprise neuroinflammatory disorders such as MS, Alzheimer's disease and Parkinson's disease. Neuroinflammatory disorders are characterized by chronic inflammation of the CNS including the brain and spinal cord and may be due to, e.g., an autoimmune reaction, aging, infectious agents (e.g., bacteria, viruses) and/or trauma. During acute inflammatory episodes of the CNS, microglial cells within the CNS are activated to respond to the source of the neural injury or insult.

Without wishing to be bound by any particular theory, typically, the BBB comprised of endothelial cells joined to each other by tight junctions, a thick basement membrane, pericytes and astrocytes, limits the passage of molecules, including immune cells, from the peripheral blood into the extracellular fluid of the CNS. However, over time as inflammation becomes chronic, the BBB may be compromised such that circulating immune cells are able to pass through to the CNS and directly interact with neurons, glial cells (microglial, astrocytes) and endothelial cells.

In some embodiments, the present disclosure provides the insight that mast cell metabolites may contribute to modulation or enhanced permeability of the blood brain barrier, which allows, inter alia, influx of immune cells (e.g., T cells, mast cells) into the CNS. In some embodiments, the present disclosure provides that influx of immune cells (e.g., T cells, mast cells) into the CNS is via lymphatic vessels within the CNS. In some embodiments, the present disclosure provides that mast cell metabolites may contribute to modulation or enhanced permeability of lymphatic vessels.

In some embodiments, the present disclosure provides enhanced permeability of the lymphatic vessels results in localization of T cells and/or mast cells in the CNS, and in particular at or near neurons. In some embodiments, the present disclosure provides enhanced permeability of the BBB results in localization of T cells and/or mast cells in the CNS, and in particular at or near neurons. In some embodiments, the present disclosure provides the insight that influx of immune cells into the CNS results in initiation, exacerbation or relapse of a neuroinflammatory and/or neurodegenerative disease, disorder and/or condition. In one embodiment, relapse is MS relapse.

In some embodiments, immune cells that enter the CNS may include B cells, T cells, macrophages, plasma cells, and mast cells. In some embodiments, such cells perpetuate inflammation through further release of cytokines and recruitment of additional immune cells through the BBB. In some embodiments, chronic neuroinflammatory response leads to eventual neurodegeneration characterized by demyelination, formation of plaques, formation of neurofibrillary tangles, etc. Chronic inflammation of the CNS is typically associated with neurodegenerative diseases such as MS, Alzheimer's Disease and Parkinson's Disease.

In some embodiments, the present disclosure teaches that a mast cell biomarker as described herein may be a useful biomarker for inflammatory disease. For example, in some embodiments, in accordance with the present disclosure, such a mast cell biomarker that may be used as an indicator for inflammatory disorder, risk of developing an inflammatory disorder, response to a therapeutic intervention, likelihood of inflammatory disorder progression and/or severity of inflammatory disorder is determined and/or detected.

Multiple Sclerosis

Among other things, the present disclosure teaches that mast cell activity may be associated with and/or characteristic of one or more features of MS. Thus, in accordance with some embodiments of the present disclosure, MS may be considered to be a mast cell activity-associated disorder.

MS is an autoimmune disorder affecting the CNS and characterized by variable symptoms such as fatigue, gait difficulties, numbness or tingling of the body, muscle weakness, dizziness, vertigo, pain, spasticity of muscles and vision problems. Over 400,000 persons in the U.S. are affected with the disorder. In MS, the myelin coating around the nerve fibers of the CNS and the nerve fibers themselves are damaged resulting in disruption in nerve transmission between the brain, spinal cord and periphery. The severity and location of the damage varies among patients resulting in variable symptoms.

Considerable evidence has demonstrated that MS is initially an immune initiated disorder, which results in secondary demyelination and axonal damage within the CNS. MS is characterized by relapses or attacks during the first 10-15 years of disease, caused by waves of inflammatory cells entering the CNS. Following this phase, many patients experience slow progressive worsening, termed the “secondary progression” which is likely due to inflammatory and neurodegenerative mechanisms centered within the CNS proper. The overall goal of MS treatment is to prevent relapses or attacks, and to slow or prevent the onset of secondary progression.

Four different types, or courses, of MS have been identified: clinically isolated syndrome (CIS), relapsing-remitting MS (RRMS), primary progressive MS (PPMS), and secondary progressive MS (SPMS). CIS is characterized by an initial episode of CNS inflammation and demyelination with neurological symptoms. The initial episode can last up to 12 hours and may or may not be followed by further episodes and development of MS. RRMS is the most common type of MS and is characterized by episodes or attacks in which new or increased severity of neurological systems are observed. During the episodes or attacks, new lesions may be observed on MRI. Between attacks (i.e., during periods of remission) the symptoms may resolve entirely or persist and become permanent. Unlike RRMS, patients with PPMS experience worsening neurologic function and accumulating disability without periods of remission. As the disease progresses, the patient may experience active disease (i.e., occasional relapses), not active disease, progressive disease with worsening symptoms and disability or without progression. Lastly, SPMS describes the disease course in which there is a progressive worsening of neurologic function, similar to what is observed in PPMS, but following an initial course of relapsing and remitting MS. In SPMS, there may be occasional relapses as well as periods of instability.

The cause of MS is not fully known but is believed to be due to the interaction of at least several factors including: immunological, environmental, infectious and/or genetic factors. Over 200 MS susceptibility genes have been identified, indicating MS is more common in a genetically susceptible host, however there is considerable evidence that environmental factors contribute, and potentially interact, to cause the onset of disease. Many of the same factors including vitamin D levels and smoking are related to the initiation of relapses, indicating a common pathophysiology in the initiation of waves of inflammatory cell activation and migration into the CNS. Without wishing to be bound by any particular theory, a key step in this process is the transmigration of immune cells into the central nervous system and in particular activated T cells. Activated T cells enter the CNS through blood vessels and directly attack myelin, secrete factors (e.g., granulocyte-macrophage colony-stimulating factor, etc.) that damage axons and recruit additional immune cells (e.g., myeloid cells, macrophages, etc.) that contribute to inflammation. The etiology of T cell activation in MS is not fully understood. There is a need to in the art to determine the etiology of T cell activation in MS patients, biomarkers of T cell activation, biomarkers of relapse of MS and biomarkers for predicting the severity of MS.

In some embodiments, the present disclosure teaches that a mast cell biomarker as described herein may be a useful biomarker for MS. For example, in some embodiments and in accordance with the present disclosure, such a mast cell biomarker that may be used as an indicator for MS, risk of developing MS, response to a therapeutic intervention, likelihood of MS progression and/or severity of MS and/or susceptibility to MS relapse is determined and/or detected.

MS and Allergy

The above evidence may lead to the postulation that mast cell degranulation caused by environmental allergies may trigger MS. Two studies have investigated this relationship. A case-control study of 195 adult MS cases and controls demonstrated an inverse relationship between respiratory tract allergies and food allergies with MS risk. No relationship was demonstrated with cutaneous allergies (Sahraian et al., Clin. Neurol. Neurosurg. (2013) 115:2099-2102). Similarly, a case-control study of pediatric MS patients demonstrated an inverse relationship of environmental allergies (12.8% vs. 20.4%, p=0.013) and food allergies (5.2% vs. 9.4%, p=0.05) compared to pediatric control subjects. No statistically significant difference in allergies to antibiotics (5.9% vs. 3.3%, p=0.161) or in incidence of allergic reactions (27.7% vs. 29.7%, p=0.591) was observed (Neiderer et al., Neurology (2016) Apr. 16, 2016, P1.380).

A further study investigated relapse rate in patients with and without environmental allergies (Diaz-Cruz et al., Neurology (2016) April 176, 2016, P2.187). Patients reporting any food allergy had a higher adjusted relapse rate (ARR) than never allergic patients (0.2398 vs. 0.1893; Wald p=0.048). No significant differences in the ARR were found when comparing the environmental (p=0.1255) or drug (p=0.4339) groups with the never allergic group, however a trend to a higher relapse rate was noted in these groups.

Alzheimer's Disease

Among other things, the present disclosure teaches that mast cell activity may be associated with and/or characteristic of one or more features of Alzheimer's Disease (AD). Thus, in accordance with some embodiments of the present disclosure, AD may be considered to be a mast cell activity-associated disorder.

Amyloid beta (Aβ) peptide 25-35 causes rapid degranulation of cultured mast cells through a pannexinl hemichannel dependent mechanism (Haracha, et al., J. Neurosci. (2015) 35:9526-9538). In AD animal models, Aβ peptide 25-35 promotes both connexin 43 and pannexinl hemichannel dependent mast cell dye uptake and histamine release, suggesting a role for mast cells in pathogenesis of the disease. Mastinib, is a selective oral tyrosine kinase inhibitor, which effectively inhibits the survival, migration and activity of mast cells. A phase II study in AD using mastinib showed some benefit in slowing cognitive decline in AD patients (Piette et al., Alzheimers Res. Ther. (2011) 3:16).

In some embodiments, the present disclosure teaches that a mast cell biomarker as described herein may be a useful biomarker for AD. For example, in some embodiments and in accordance with the present disclosure, such a mast cell biomarker that may be used as an indicator for AD, risk of developing AD, response to a therapeutic intervention, likelihood of AD progression and/or severity of AD are determined and/or detected.

Migraine

Among other things, the present disclosure teaches that mast cell activity may be associated with and/or characteristic of one or more features of migraine. Thus, in accordance with some embodiments of the present disclosure, migraine may be considered to be a mast cell activity-associated disorder.

Without wishing to be bound by any particular theory, mast cells located in the dura mater have been purported to play a key role in triggering pain nociceptors in meningeal blood vessels. Serotonin, prostacyclin (PGI2), and to a lesser extent, histamine and tryptase, are likely to serve as the mediators through which dural mast cells promote the activation of meningeal nociceptors (Levy et al., Curr. Pain Headache Rep. (2009) 13:237-240; Zhang, et al., J. Pharmacol. Exp. Ther. (2007) 322:806-812). Pituitary adenylate cyclase activating peptide 38 (PACAP38) has been associated with induction of migraine, and triggering of mast cell degranulation (Baun et al., Cephalagia (2012) 32:337-345). Interestingly, animal models have demonstrated that the density of dural mast cells in females fluctuates during the estrous cycle and is overall higher than in males, and highlights a role for estrogen induced activation or migration of mast cells (Boes et al., Cephalalgia (2012) 32:924-931).

In some embodiments, the present disclosure teaches that a mast cell biomarker as described herein may be a useful biomarker for migraine. For example, in some embodiments and in accordance with the present disclosure, such a mast cell biomarker that may be used as an indicator for migraine, risk of developing migraine, response to a therapeutic intervention, likelihood of migraine progression and/or severity of migraine is determined and/or detected.

Autoimmune and Autoimmune Related Disorders

Among other things, the present disclosure teaches that mast cell activity may be associated with and/or characteristic of one or more features of autoimmune and autoimmune related diseases, disorders and/or conditions. Thus, in accordance with some embodiments of the present disclosure, autoimmune and autoimmune related diseases, disorders and/or conditions may be considered to be a mast cell activity-associated disorder.

In some embodiments, autoimmune and autoimmune related diseases, disorders and/or conditions that may be a mast cell activity-associated disorder in accordance with the present disclosure may be or comprise, for example, acute disseminated encephalomyelitis, acute hemorrhagic leukoencephalitis, Addison's Disease, agammaglobulinemia, alopecia areata, amyotrophic lateral sclerosis, ankylosing spondylitis, anti-GBM/TBM nephritis, anti-magigm peripheral neuropathy, antiphospholipid syndrome, antisynthetase syndrome, asthma, atopic allergy, atopic dermatitis, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune pancreatitis, autoimmune peripheral neuropathy, autoimmune polyendocrine syndrome, autoimmune progesterone dermatitis, autoimmune thrombocytopenic purpura, autoimmune urticarial, autoimmune uveitis, Balo disease/Balo concentric sclerosis, Bechets Syndrome, Berger's disease, Bickerstaffs encephalitis, Blau syndrome, Bullous pemphigoid, Castleman's disease, Celiac disease, Charcot-Marie-Tooth syndrome, Chronic Fatigue Syndrome (CFS), chronic inflammatory demyelinating polyneuropathy, chronic recurrent multifocal osteomyelitis, Crohn's disease, irritable bowel syndrome, Churg-Strauss syndrome, cicatricial pemphigoid, Cogan syndrome, cold agglutinin disease, complement component 2 deficiency, cranial arteritis, CREST syndrome, Cushing's Syndrome, cutaneous leukocytoclastic angiitis, Dego's disease, Dercum's disease, dermatitis herpetiformis, dermatomyositis, diabetes mellitus type 1, diffuse cutaneous systemic sclerosis, discoid lupus erythematosus, Dressler's syndrome, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, fibrodysplasia ossificans progressive, fibromyalgia, fibrosing aveolitis, gastritis, gastrointestinal pemphigoid, giant cell arteritis, glomerulonephritis, Goodpasture's syndrome, Graves' disease, Guillain-Barré syndrome (GBS), haemolytic anaemia, Hailey-Hailey Disease, Hashimoto's encephalitis, Hashimoto's thyroiditis, Henoch-Schonlein purpura, Herpes gestationis, HIV, hypogammaglobulinemia, idiopathic inflammatory demyelinating diseases, idiopathic pulmonary fibrosis, idiopathic thrombocytopenic purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Lyme disease, Majeed syndrome, Meniere's disease, microscopic polyangiitis, Miller-Fisher syndrome, mixed connective tissue disease, morphea, Mucha-Habermann disease, MS, myalgic encephalomyelitis, myasthenia gravis, myositis, neuromyelitis optica (also known as Devic's Disease), neuromyotonia, occular cicatricial pemphigoid, opsoclonus myoclonus syndrome, Ord thyroiditis, Parkinson's disease, Palindromic rheumatism, pediatric autoimmune neuropsychiatric disorders associated with Streptococcus (PANDAS), paraneoplastic cerebellar degeneration, paroxysmal nocturnal hemoglobinuria, Parry Romberg syndrome, pars planitis, Parsonnage-Turner syndrome, pemphigus, pemphigus vulgaris, perivenous encephalomyelitis, pernicious anaemia, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pure red cell aplasia, pyoderma gangrenosum, Rasmussen's encephalitis, Raynaud phenomenon, Reiter's syndrome, relapsing polychondritis, restless leg syndrome, retroperitoneal fibrosis, rheumatoid arthritis, rheumatoid fever, sarcoidosis, Schmidt syndrome, Schnitzler syndrome, scleritis, scleroderma, Sjögren's syndrome, spondyloarthropathy, stiff person syndrome, Still's disease, subacute bacterial endocarditis, Susac's syndrome, Sweet's syndrome, Sydenham chorea, sympathetic ophthalmia, Takayasu's arteritis, temporal arteritis (also known as giant cell arteritis), Tolosa-Hunt syndrome, transverse myelitis, ulcerative colitis, undifferentiated connective tissue disease, undifferentiated spondyloarthropathy and vasculitis vitiligo.

In some embodiments, the present disclosure teaches that a mast cell biomarker as described herein may be a useful biomarker for autoimmune and autoimmune related diseases, disorders and/or conditions. For example, in some embodiments and in accordance with the present disclosure, such a mast cell biomarker may be used as an indicator for autoimmune and autoimmune related diseases, disorders and/or conditions, risk of developing autoimmune and autoimmune related diseases, disorders and/or conditions, response to a therapeutic intervention, likelihood of autoimmune and autoimmune related diseases, disorders and/or conditions progression and/or severity of autoimmune and autoimmune related diseases, disorders and/or conditions is determined and/or detected.

Stroke

In some embodiments, mast cell activity-associated disorders include stroke. Mast cell deficient mice exhibit fewer cells in the granulocyte and macrophage population (CD11bhigh CD45high cells) at 3 days after stroke than do their corresponding wild type mice (Arac et al., Am. J. Pathol. (2014) 184:2493-2504). Engraftment of mast cells into the meninges of the mast cell deficient mice restores the same phenotype as is observed in the wild-type mice. Increased granulocyte and macrophage populations are associated with increased stroke size and severity. Furthermore, knockout of mast cell produced IL-6 restores the milder stroke phenotype.

Mast Cells

Mast cells, or mastocytes, are granulocytes derived from myeloid lineage which circulate in the blood as precursor cells and mature in peripheral tissues. Mast cells are related to basophils, and share a common precursor in the bone marrow, termed CD34 cells. The primary growth factor for mast cell development is stem cell factor (SCF), also known as the ligand for c-kit, which is expressed on the mast cell surface. Mast cells are characterized by abundant intracellular granules comprising pre-formed inflammatory metabolites (e.g., prostaglandins, leukotrienes, histamine and cytokines). Unlike other monocytes, mast cells survive for weeks to months and have some proliferative potential following differentiation (Galli et al., Annu. Rev. Immunol. (2005), 23:749-786).

Mast cells are present in most tissues, including connective tissue, the brain and spinal cord, and localize around blood vessels, lymphatics and nerves. Within the brain, mast cells are present in areas that mediate visceral sensory or neuroendocrine functions (e.g., pituitary stalk, pineal gland, thalamus and hypothalamus) or at the blood-brain barrier or blood-cerebrospinal fluid barrier (e.g., postrema, choroid plexus, meninges). They are commonly found in tissues that interface with the external environment (e.g., skin, mucosa of the digestive system, mucosa of the pulmonary system, genitourinary tracts, conjunctiva) which is highly relevant to initiation and propagation of immune responses (Bischoff et al., Nat. Rev. Immunol. (2007) 7:93-104).

Mast cells play a role in inflammatory processes, such as hypersensitivity and allergic reaction, through degranulation whereby the cells are stimulated to release mast cell metabolites, many of which are mediators of inflammation. In some embodiments, mast cell metabolites can be categorized as preformed granule associated metabolites or lipid derived metabolites. In some embodiments, preformed granule associated metabolites include, for example, serine proteases (e.g., tryptase, chymase, carboxypeptidase A3, cathepsin G), histamine, proteoglycans (e.g., heparin, chondroitin sulphate), cytokines (e.g., interleukins (e.g., IL-1, IL-2, IL-3, IL-4, IL-5, IL-6, IL-8), TNF-α, granulocyte macrophage colony-stimulating factor (GM-CSF), macrophage inflammatory protein alpha (MIP-1α), macrophage inflammatory protein beta (MIP-1β), interferon-gamma (INF-γ)) and growth factors (e.g., vascular endothelial growth factor A). In some embodiments, lipid derived metabolites include, for example, thromboxanes, prostaglandins (e.g., prostaglandin D₂, prostaglandin E₂), leukotrienes (e.g., leukotriene C4), platelet-activating factor).

Mast cell stimulation results in expression of genes encoding immune modulators such as chymase, TNF-α, CXCL2, proteases and IL-1(3. Mast cells are able to transfer the preformed granules to adjacent cells of the immune system and nervous system by transgranulation via pseudopodia (Wilhelm et al., Eur. J. Neurosoci. (2005) 22:2238-2248).

Stimulation of mast cell activity and/or degranulation occurs through a number of mechanisms including binding of immunoglobulins. In some embodiments, mast cells express a high-affinity receptor for the Fc region of IgE. During allergic reactions, IgE, which binds to mast cells, bind an allergen causing the mast cells to degranulate. In some embodiments, mast cells are activated through other receptors including those that bind IgG, cytokines, complement, neuropeptides, hormones, and heat shock proteins. In some embodiments, degranulation and the resulting efflux of active mast cell metabolites into the surrounding tissues results in, among other things, increased permeability of blood vessels, increased permeability of lymphatic vessels, contraction of smooth muscles and increased mucus production.

Mast cells express receptors that allow for differential activity, including differential release of mast cell metabolites. For example, mast cells express receptors for cytokines, chemokines, complement component 3a, complement component C5a and pathogen-associated molecular patterns (PAMPs) (Costanza et al., Int. J. Mol. Sci. (2012) 13:15107-15125). In some embodiments, mast cells bind a ligand that stimulates mast cell activity important in the pathogenesis of mast cell activity-associated disorders. In some embodiments, mast cell activity is degranulation and release of mast cell metabolites. In some embodiments, mast cell metabolites increase permeability of the blood brain barrier. In some embodiments, mast cell metabolites activate T cells to breakdown myelin. In some embodiments, mast cell metabolites increase permeability of the lymphatic vessels. In some embodiments, increase in permeability of lymphatic vessels results in localization of T cells and/or mast cells in the CNS, and in particular at or near the myelin sheath of neurons. In addition to playing a key role in allergy and anaphylaxis, mast cells are involved in wound healing, angiogenesis, immune tolerance, defense against pathogens and BBB function.

In some embodiments, the present disclosure provides the insight that mast cell activity, may contribute to enhanced localization of T cells and/or mast cells at CNS sites. In some embodiments, enhanced localization of T cells and/or mast cells at CNS sites results from, for example, enhanced permeability of the blood brain barrier through increased permeability of blood vessels present in the CNS, which allows, inter alia, influx of immune cells (e.g., T cells, mast cells) into the CNS. In some embodiments, the present disclosure provides the insight that mast cells that are activated are mast cells present in the meninges. In some embodiments, the present disclosure provides the insight that mast cells that are activated are mast cells present in the pia mater and the dura matter.

Mast cells are able to influence immune response by acting as antigen presenting cells and expressing Class I and Class II MHC molecules, by activating T cells, by enhancing B cell proliferation and activation and by stimulating isotype switching (Russi et al., Clin. Immunol. (2016), in press).

### Mast Cells and MS

In some embodiments, the present disclosure defines biological pathways not previously understood to be implicated in initiation, development, maintenance and/or relapse of MS and/or another inflammatory disease, disorder and/or condition (e.g., neuroinflammatory disease, disorder and/or condition). In some embodiments, the biological pathway not previously understood to be implicated in initiation, development, maintenance and/or relapse of MS and/or another inflammatory disease, disorder and/or condition is regulation (e.g., increase, decrease or modulation) of BBB permeability by, or through, mast cell activity. In some embodiments, the biological pathway not previously understood to be implicated in initiation, development, maintenance and/or relapse of MS and/or another inflammatory disease, disorder and/or condition is regulation (e.g., increase, decrease or modulation) of immune cell (e.g., T cells, mast cells) localization by, or through, mast cell activity. In particular embodiments, T cells are localized to the CNS by, or through, mast cell activity.

Mast cells are present in the brain and spinal cord, and localize around blood vessels, lymphatics and nerves. Within the brain, mast cells are present in areas that mediate visceral sensory or neuroendocrine functions or at the blood-brain barrier or blood-cerebrospinal fluid barrier (e.g., postrema, choroid plexus, dural layer of the meninges). In some embodiments, the population of mast cells that reside in the meninges may play a role in the regulation of blood brain barrier permeability. A recent discovery is the presence of lymphatics within the meningeal layer of the brain, adjacent to the dural sinuses (Louveau et al., Nature (2015) 523:337-341). These structures express all of the molecular hallmarks of lymphatic endothelial cells, have been shown to be able to carry both fluid and immune cells from the cerebrospinal fluid, and are connected to the deep cervical lymph nodes. Mast cells are reported to be present adjacent to these lymphatic vessels, and may play a key role in guiding the recruitment and education of other immune cells including T cells (Louveau et al., Trends Immunol. (2015) 36:569-577).

Studies in animal models of chronic as well as relapsing multiple sclerosis (i.e., experimental autoimmune encephalomyelitis or EAE) have demonstrated that mast cell deficient mice exhibit significantly reduced disease severity, but retain the relapsing-remitting course, which is reversed by selective reconstitution of mast cells (Piconese et al., Lab. Investig. (2011) 91:627-641; Sayed, et al., J. Immunol. (2011) 186:3234-3298; Secor et al., J. Exp. Med. (2000) 191: 813-822). Most of these studies, including more recent works suggest that EAE can proceed in the absence of mast cells, however disease severity may be reduced. (Sayed, et al., J. Immunol. (2011) 186:8234-3298; Bennett et al., J. Immunol. (2009) 182:5507-5514). Studies have shown that meningeal mast cells are activated within hours of induction and resulting in the expression of genes encoding chymase, TNF-α, CXCL2, proteases and IL-1β (Christy et al. J. Autoimmun. (2013) 42:50-61). Furthermore, there is evidence of mast cell degranulation in MS, and high levels of tryptase and histamine, two compounds released by mast cells upon degranulation, can be found in the CSF of MS patients (Ibrahim et al., J. Neuroimmunol. (1996) 70:131-138; Rozniecki et al., Ann. Neurol. (1995) 37:63-66). Mast cells, as well as increased expression of genes encoding histamine 1 receptor, tryptase and FcεRIα, have been demonstrated in the white matter plaques of patients with MS (Lock et al., Nat. Med. (2002) 8:500-508).

Secondary Effects of Mast Cells on MS Immune Cells

In some embodiments, mast cells may exert effects on other CNS immune cell populations. One study demonstrated that IL-1β expression by mast cells promotes GM-CSF expression by T cells, which is essential for T cell encephalitogenicity (Russi et al., Clin. Immunol. (2016) in press). Microglia have been implicated in the pathogenesis of progressive disease. Mast cells have been shown to induce microglia activation and pro-inflammatory metabolite release via PAR-2-MAPK-NF-kappa B signaling pathways (Zhang et al., Cell Physiol. Biochem. (2012) 29:931-940). More recently, the role of stress responses through corticotropin releasing hormone (CRH), in the activation of mast cells, has been postulated (Esposito et al., Brain Res. (2001) 888:117-127; Esposito et al., J. Pharmacol. Exp. Ther. (2002) 303:1061-1066).

Mast Cells in MS Lesions

Studies in multiple sclerosis patients have identified mast and mast cell transcripts within MS brain lesions, including in perivascular infiltrates (Toms et al., J. Neuroimmunol. (1990) 30:169-177; Olsson, Acta Neurol. Scand. (1974) 50:611-618; Ibrahim et al., J. Neuroimmunol. (1996) 70:131-138; Couturier et al., J. Neuroimmunol. (2008) 195:176-185). Cerebrospinal fluid collected from MS patients has demonstrated elevated levels of tryptase, which is a proteolytic enzyme produced by mast cells, (Rozniekci et al., Ann. Neurol. (1995) 37:63-66) suggesting that mast cell products may be involved in MS.

Without wishing to be bound by any particular theory, the present disclosure proposes that mast cell activity or factors as described herein may contribute to one or more of increased traversal, or transmigration, of immune cells (e.g., T cells, mast cells) from the circulatory and/or lymphatic system. In some embodiments, increased traversal of immune cells (e.g., T cells, mast cells) from the circulatory and/or lymphatic system results from modulation of permeability (at least with respect to immune cells) of blood vessels and/or lymphatic vessels, which allows, inter alia, influx of immune cells (e.g., T cells, mast cells) into the CNS and targeting of immune cells (e.g., T cells, mast cells) to one or more particular CNS sites (e.g., to the myelin sheath, to the basal lamina of the BBB, to lymphatic vessels). In some embodiments, mast cell activity may include any one, or a combination of, degranulation, transgranulation, a change in level and/or location of a mast cell activity factor, detecting level of a mast cell activity factor gene product, localization or migration of mast cells within a tissue or organ, degree or type of interaction with other cell types, degree or type of interaction with one or more particular proteins (e.g., myelin) or sites (e.g., basal lamina of the BBB).

In one embodiment, interaction with other cell types is activation of encephalitogenic T cells. In some embodiments, mast cell activity within the dural layer, contributes to initiation and/or propagation of MS relapses through activation of encephalitogenic T cells.

### Mast Cells and the Lymphatic System

The CNS is an immune privileged site and no peripheral immune cells are found within the healthy parenchyma. However, the borders of the CNS (e.g., the meningeal linings) contain a high number of immune cells. The recently discovered meningeal lymphatics that drain the CNS ensure meningeal immunity to interact with the periphery through a direct connection to the deep cervical lymph nodes.

Meningeal immunity plays a major role in brain function in health and disease. In the mouse model of MS, EAE, the meninges have a central role in the regulation of neuroinflammation. Evidence suggests that inflammatory processes start in the meninges, with peripheral immune cells (e.g., T cells) extravasating through meningeal blood vessels. Prior to the present invention, the mechanism by which immune cells (e.g., T cells) are activated and enabled to penetrate the pia matter and proceed to attack the brain parenchyma, had not been recognized.

The present disclosure encompasses the insight that mast cells and/or mast cell metabolites, found within the meninges, alter the phenotype of meningeal immune cells (e.g., T cells) and affect their ability to attack the CNS parenchyma, and in particular myelin. In some embodiments, mast cells may alter the phenotype of meningeal T cells by degranulation and triggering an immune process. The present disclosure recognizes that immune cells activated by mast cell activity express unique biomarkers of activation. In some embodiments, an immune cell is an activated T cell.

In some embodiments, the present disclosure defines biological pathways not previously understood to be implicated in initiation, development, maintenance and/or relapse of MS and/or another inflammatory disease, disorder and/or condition. In some embodiments, the biological pathway not previously understood to be implicated in initiation, development, maintenance and/or relapse of MS and/or another inflammatory disease, disorder and/or condition is contribution by mast cell activity factors to enhanced localization of T cells at CNS sites. Without wishing to be bound by any particular theory, the present disclosure proposes that mast cell activity or factors as described herein may contribute to one or more of increased traversal of immune cells (e.g., T cells, mast cells) from the lymphatic system, for example by increasing permeability (at least with respect to immune cells) of lymphatic vessels, which allows, inter alia, influx of immune cells (e.g., T cells, mast cells) into the CNS and targeting of immune cells (e.g., T cells, mast cells) to one or more particular CNS sites (e.g., to the myelin sheath, to the basal lamina of the BBB). In some embodiments, mast cell activity may include any one, or a combination, of degranulation, transgranulation, a change in level and/or location of a mast cell activity factor, detecting level of a mast cell activity factor gene product, localization or migration of mast cells within a tissue or organ, degree or type of interaction with other cell types, degree or type of interaction with one or more particular proteins (e.g., myelin) or sites (e.g., basal lamina of the BBB). In some embodiments, degranulation occurs in the dural layer of the meninges. In one embodiment, interaction with other cell types is the activation of encephalitogenic T cells. In some embodiments, degranulation of mast cells within the dural layer, contribute to initiation and/or propagation of MS relapses through activation of encephalitogenic T cells.

### Biomarkers of Mast Cell Activity-Associated Disorders

Among other things, the present disclosure defines one or more biomarkers of mast cell activity-associated disorders. That is, the present disclosure defines mast cell activity biomarkers and establishes their relevance to certain diseases, disorders and/or conditions as described herein. As noted above, in some embodiments, a mast cell biomarker may be or comprise one or more of degranulation, transgranulation, mast cell metabolites, mast cell activity factors, a change in level and/or location of a mast cell activity factor, localization or migration of mast cells within a tissue, organ or mast cell activity site, degree or type of interaction between mast cells and other cell types, degree or type of interaction between mast cells and particular proteins or mast cell activity sites, etc. The present disclosure identifies the source of a problem in prior work to identify useful biomarkers in that such efforts typically focused on the wrong biological pathways, events, and/or timing. The present disclosure teaches relevance and import of certain events associated with mast cell activity and/or degranulation to MS and/or to one or more other inflammatory diseases, disorders and/or conditions, and provides technologies that define biomarkers for such events.

In particular embodiments, the present disclosure defines one or more biomarkers that correlate with susceptibility to MS relapse. Once defined, such biomarkers may be used in methods of assessing relapse risk in an MS patient by detecting one or more mast cell activity factors or biomarkers in a sample from a patient. Alternatively or additionally, such biomarkers may be useful for assessing or predicting severity of relapse in MS patients.

In some embodiments, a biomarker may be or comprise an entity or event whose presence, level, location, or form, correlates with a particular biological event or state of interest, so that it is considered to be a “marker” of that event or state (e.g., a mast cell activity-associated disorder). In some embodiments, a biomarker can be used as an indicator or a disease, risk of developing a disease, carrier status, response to a therapeutic intervention, likelihood of disease progression and/or severity of a disease. A biomarker may comprise a single marker (e.g., metabolite, mast cell metabolite) or may comprise more than one marker (e.g., a plurality of markers). In some embodiments, a biomarker comprises the presence and/or absence of a marker. In some embodiments, a biomarker comprises a level of a marker. In some embodiments, a biomarker comprises the presence and/or absence of a plurality of markers, particular levels of a plurality of markers or a combination thereof. In some embodiments, a biomarker may be a profile or a pattern of biomarkers. Typically, a suitable biomarker has a characteristic that can be objectively measured and evaluated as an indicator. In some embodiments, a biomarker may be a mast cell activity biomarker. In some embodiments, a biomarker may be a mast cell activity factor.

A biomarker may be differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney, odds ratio, Linear Discriminant Analysis, Quadratic Discriminant Analysis and K-nearest neighbor. Biomarkers, alone or in combination, provide measures of relative risk that a subject belongs to one phenotypic status or another. Therefore, they are useful as biomarkers for disease (diagnostics), therapeutic effectiveness of a drug (theranostics) and drug toxicity. For example, a suitable biomarker for mast cell activity-associated disorders is differentially expressed between patients with mast cell activity-associated disorders and healthy individuals.

In some embodiments, “differential expression profiling” may be used to identify biomarkers for mast cell activity-associated disorders. As used herein, the term “differential expression profiling” refers to methods of comparing the gene, protein or lipid-derived metabolite expression levels or patterns in two or more samples (e.g., samples obtained from patients with mast cell activity-associated disorders vs. samples obtained from healthy control individuals). Typically, a gene, protein or lipid-derived metabolite is differentially expressed if the difference (e.g., increase or decrease) in the expression level or pattern between two samples is statistically significant (i.e., the difference is not caused by random variations). In some embodiments, a gene, lipid-derived metabolite or protein is differentially expressed if the difference in the expression level between two samples (e.g., a biological sample and a reference or control sample) is more than 20%, 30%, 40%, 50%, 60%, 70%, 80%, 90%, 1-fold, 1.2-fold, 1.5-fold, 1.75-fold, 2-fold, 2.25-fold, 2.5-fold, 2.75-fold, 3-fold, 4-fold, 5-fold, 10-fold or 100-fold.

Exemplary mast cell activity factors for mast cell activity-associated disorders according to the present invention are listed in Table 1. In some embodiments, a mast cell activity biomarker may be or comprise a mast cell activity factor, or marker, of mast cell activity (e.g., activation, degranulation, migration, binding, etc.). In some embodiments, a mast cell activity biomarker may be or comprise a mast cell activity factor for diagnosing a mast cell activity-associated disorder, or for likelihood that a mast cell activity-associated disorder, may develop. In some embodiments, a mast cell activity biomarker may be or comprise a mast cell activity factor for a particular type of mast cell activity-associated disorder, such as cancer, an inflammatory disorder, a neuroinflammatory disorder or an autoimmune disorder. In some embodiments, a mast cell activity biomarker may be or comprise a mast cell activity factor for a therapeutic outcome, or likelihood thereof. Thus, in some embodiments, a mast cell activity biomarker may be or comprise a mast cell activity factor that is predictive of development of a mast cell activity-associated disorder. In some embodiments, a mast cell activity biomarker may be or comprise a mast cell activity factor that is prognostic of a mast cell activity-associated disorder. In some embodiments, a mast cell activity biomarker may comprise a mast cell activity factor that can be used to predict the severity of a mast cell activity-associated disorder or recurrence of a mast cell activity-associated disorder. In some embodiments, a mast cell activity biomarker may comprise a mast cell activity factor that can be used to predict MS relapse.

In some embodiments, individual mast cell activity factors or mast cell activity biomarkers described herein may be used. In some embodiments, at least two, three, four, five, six, seven, eight, nine, ten, eleven, twelve, thirteen, fourteen, fifteen, sixteen, seventeen, eighteen, nineteen or more mast cell activity factors selected from Table 1 may be used in combination as a panel.

In some embodiments, inventive mast cell activity biomarkers described herein may be used in conjunction with one or more additional markers, in particular, those markers known to be associated with mast cell activity-associated disorders. In some embodiments, a mast cell activity biomarkers described herein may be used in conjunction with one or more additional markers, in particular, those markers known to be associated with neuroinflammatory disorders, and in particular MS. In some embodiments, a one or more additional biomarkers include clinical findings, MRI findings and/or evoked potential testing.

A mast cell activity biomarker, or a mast cell activity factor, may be an entity of any chemical class. For example, in some embodiments, a mast cell activity biomarker, or factor, may be or comprise a nucleic acid, a polypeptide, a lipid, a carbohydrate, a small molecule, an inorganic agent (e.g., a metal or ion), or a combination thereof. In some embodiments, a mast cell activity biomarker, or factor, may be or comprise a serine protease, a proteoglycan, a cytokine, a growth factor, a chemokine, a nucleic acid, an immunoglobulin or a lipid derived mast cell metabolite as provided in Table 1.

In some embodiments, a mast cell activity biomarker, or factor, may be or comprise a cell surface marker, for example a cell surface marker present on a T cell. In some embodiments, a mast cell activity biomarker, or factor, may be or comprise a cell surface marker present on a B cell, a macrophage, a neuron, an astrocyte, a microglial cell, a pericyte and/or an endothelial cell of the blood brain barrier. In some embodiments, a cell surface marker is a receptor. In some embodiments, a cell surface marker is a Class I and Class II MHC molecule.

In some embodiments, a mast cell activity biomarker, or factor, is intracellular. In some embodiments, a mast cell activity biomarker, or factor, may be or comprise a mast cell metabolite present in a preformed granule. In some embodiments, a mast cell activity biomarker, or factor, may be or comprise a lipid derived mast cell metabolite.

In some embodiments, a mast cell activity biomarker, or factor, may be or comprise a nucleic acid, for example an RNA or a messenger RNA (mRNA). In some embodiments, the messenger RNA profile expressed during mast cell activity or activation, correlates with a particular mast cell activity-associated disorder. In some embodiments, the messenger RNA profile expressed during mast cell activity or activation is useful for diagnosis of a particular mast cell activity-associated disorder. In some embodiments, messenger RNA may be expressed from one or more genes encoding a mast cell metabolite of mast cells. In some embodiments, messenger RNA may be expressed from a gene encoding c-kit (also known as CD117 or mast/stem cell growth factor receptor). In some embodiments, messenger RNA may be expressed from a gene encoding a member of the P450 family of enzymes, for example, messenger RNA may be expressed from a gene encoding CYP21A2 (SEQ ID NO:1).

In some embodiments, a biomarker may be an allelic variant of a gene associated with a mast cell activity-associated disorder, for example a gene member of the P450 family (e.g., CYP21A2). In some embodiments, an allelic variant may be a point mutation that changes a single nucleotide and results in a change in an amino acid within an expressed protein. In some embodiments, a single nucleotide change is silent in that an amino acid is not changed within an expressed protein. In some embodiments, a single nucleotide change results in a change in mRNA splicing. In some embodiments, an allelic variant may be, for example, a small insertion or deletion, a gene conversion, a large insertion or a duplication. Some embodiments of the present invention provide methods of identifying genetic markers (e.g., allelic variant) that predispose an individual to the development of a mast cell activity-associated disorder (e.g., autoimmune, MS). Some embodiments of the invention provide methods of treatment comprising modification of the genetic marker (e.g., allelic variant) to prevent or treat a mast cell activity-associated disorder (e.g., autoimmune, MS).

In some embodiments, a mast cell activity biomarker, or factor, is found outside of cells (e.g., is secreted or is otherwise generated or present outside of cells). In some embodiments, a mast cell activity biomarker, or factor, may be or comprise a preformed granule associated metabolite or a lipid derived metabolite. In some embodiments, a preformed granule associated metabolite may be or comprise, for example, a serine protease. In some embodiments, a serine protease may be or comprise, for example, tryptase, chymase or carboxypeptidases. In some embodiments, a preformed granule associated metabolite may be or comprise, for example, a proteoglycan. In some embodiments, a proteoglycan may be or comprise heparin or chrondroitin sulphate. In some embodiments, a preformed granule associated metabolite may be or comprise histamine. In some embodiments, a preformed granule associated metabolite may be or comprise a cytokine. In some embodiments, a cytokine may be or comprise, for example, interleukins, TNF-α, GM-CSF, MIP-1α, MIP-1β and INF-γ. In some embodiments, an interleukin may be or comprise IL-1, IL-2, IL-3, IL-4, IL-5, IL-6, IL-8. In some embodiments, a preformed granule associated metabolite may be or comprise a growth factor. In some embodiments, a growth factor may be or comprise vascular endothelial growth factor A.

In some embodiments, a mast cell activity biomarker, or factor, may be or comprise a lipid derived metabolite. In some embodiments, a lipid derived metabolites may be, for example, thromboxanes, prostaglandins, leukotrienes and/or platelet-activating factor. In some embodiments, a prostaglandin may be prostaglandin D₂, prostaglandin E₂ and/or 11-β-prostaglandin F₂-α. In some embodiments, a leukotriene may be leukotriene C4.

In some embodiments, a mast cell activity biomarker, or factor, may comprise a metabolite of a biomarker that is released directly from a mast cell. The metabolite may be present in urine, for example, N-methylhistamine.

TABLE 1
Exemplary Mast Cell Activity Biomarkers or Factors

histamine
N-methylhistamine
chromogranin A
cytokines
Interleukin-1 (IL-1)
Interleukin-2 (IL-2)
Interleukin-3 (IL-3)
Interleukin-4 (IL-4)
Interleukin-5 (IL-5)
Interleukin-6 (IL-6)
Interleukin-8 (IL-8)
Interleukin-10 (IL-10)
Interleukin-17 (IL-17)
Interleukin-33 (IL-33)
TNF-α
TGF-β
granulocyte macrophage colony-stimulating factor (GM-CSF)
macrophage inflammatory protein alpha (MIP-1α)
macrophage inflammatory protein beta (MIP-1β)
Interferon-gamma (INFγ)
eosinophilic chemotactic factor
chemokines
CC-chemokine-ligand 2
growth factors
vascular endothelial growth factor A (VEGFA)
nerve growth factor (NGF)
proteoglycans
heparin
chondroitin sulphate
neutral proteases
tryptase
chymase
carboxypeptidase A3
cathepsin G
prostaglandins
prostaglandin D₂ (PGD₂)
prostaglandin E₂ (PGE₂)
11-β-prostaglandin F2-α (11β-PGF2α)
tetranor-prostaglandin D₂ metabolite (tetranor-PGDM)
leukotrienes
leukotriene E₄
leukotriene B₄
leukotriene C₄
immunoglobulins
immunoglobulin E (IgE)
thromboxanes
nucleic acids

### Biomarker Determination

In some embodiments, the present disclosure provides methods or systems for the identification or determination of useful biomarkers of mast cell activity-associated diseases, disorders and/or conditions. A variety of methods may be used to determine a biomarker of a mast cell activity-associated disorder. In some embodiments, the method may include the step of determining presence, level and/or location of one or more mast cell activity factors in a sample of subjects suffering from or susceptible to mast cell activity-associated disorder. In some embodiments, subjects may be newly diagnosed with the mast cell activity-associated disorder. In some embodiments, subjects may be suffering from an acute episode of the disorder or may be in a chronic phase of the disorder. In some embodiments, subjects may be suffering from a relapsing and remitting course of the disorder. In some embodiments, subjects may be suffering from CIS, RRMS, PPMS or SPMS. In some embodiments, the present disclosure provides systems for identifying one or more factors or biomarkers of a particular stage or severity of disease. In some embodiments, a sample is whole blood, plasma, serum, urine, cerebrospinal fluid or lymphatic fluid.

In some embodiments, provided methods may comprise detecting a correlation between a determined presence, level and/or location of the one or more mast cell activity factors with incidence, severity or therapeutic response of the mast cell activity-associated disorder and establishing the determined presence, level and/or location of a mast cell activity biomarker for the incidence, severity, or therapeutic response. In some embodiments, incidence, severity or therapeutic response of the mast cell activity-associated disorder correlates with the presence, level and/or location of mast cell activity, mast cell proliferation, mast cell migration, release of cytokines, release of lipid derived metabolites, release of granule-associated metabolites, hydration, inflammation and combinations thereof. In some embodiments, mast cell activity-associated disorder is MS and the incidence, severity or therapeutic response of the MS correlates with the presence, level and/or location of mast cell activity, mast cell proliferation, mast cell migration, release of cytokines, release of lipid derived metabolites, release of granule-associated metabolites, hydration, inflammation and combinations thereof.

In some embodiments, a correlated determined presence, level and/or location includes a plurality of data points, each representing presence, level and/or location of a different mast cell activity factor. In some embodiments, plurality of data points is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 50 or at least 100. In some embodiments, different mast cell activity factors may include at least 2, 3, 4, 5, 6, 7, 8, 9, 10, 11, 12, 13, 14, 15, 16, 17, 18, 19 or 20 mast cell activity factors selected from Table 1.

In some embodiments, at least one data point represents a level relative to an established threshold for a particular mast cell activity factor. In some embodiments, a particular mast cell activity factor is a mast cell activity factor selected from Table 1. In some embodiments, an established threshold is a threshold determined in a reference or control sample. In some embodiments, a reference sample is derived from a healthy subject (e.g., a subject not suffering from the mast cell activity-associated disorder). In some embodiments, a reference sample is derived from a group of healthy subjects. In some embodiments, a reference sample is derived from the subject suffering from the mast cell activity-associated disorder prior to onset of the disorder. In some embodiments, a reference sample is a historical reference. In some embodiments, at least one data point represents a level that is more than 20%, 30%, 40%, 50%, 60%, 70%, 80%, 90%, 1-fold, 1.2-fold, 1.5-fold, 1.75-fold, 2-fold, 2.25-fold, 2.5-fold, 2.75-fold, 3-fold, 4-fold, 5-fold, 10-fold or 100-fold higher or lower than the established threshold for a particular mast cell activity factor.

In some embodiments, a mouse model of a mast cell activity-associated disorder may be used to determine a mast cell activity biomarker. In some embodiments, a mouse model is a model of MS, an inflammatory disorder (e.g., neuroinflammatory disorder) a cancer or an autoimmune disease. In some embodiments, a mouse model of an autoimmune disease is the non-obese diabetic (NOD) mouse (model for Type 1 diabetes and Sjogren's Syndrome), (NZB×NZW)F1,MRL/lpr (model for systemic lupus erythematosus), experimental autoimmune encephalitis in SJL mouse (model for MS), collagen-induced arthritis in DBA/1 mouse (rheumatoid arthritis), Bcl-2 transgene mouse (model for systemic lupus erythematosus) and Apcs^{-/-} mouse (model for systemic lupus erythematosus). In some embodiments, a tissue (e.g., brain, spinal cord, nerves, lungs, muscles, liver, kidney) or body fluid (blood, lymphatic tissue, CNS, urine) of a mouse is collected and analyzed to determine a mast cell activity factor or mast cell activity biomarker. In some embodiments, a factor or biomarker is determined by visualizing T-cell activation factors, B-cell activation factors and/or mast cell degranulation factors in the tissue or body fluid obtained from a mouse.

### Detection of Biomarkers

In some embodiments, the present disclosure provides systems and/or methods for detecting, measuring and/or characterizing useful biomarkers. A variety of methods may be used to measure biomarker levels in a biological sample. Typically, any characteristic indicative of expression or activity levels for biomarkers may be used to practice the invention. In some embodiments, a protein expression level of a biomarker in a sample is measured. In some embodiments, a lipid derived mast cell metabolite expression level is measured. In some embodiments, a nucleic acid expression level of a biomarker in a sample is measured. In some embodiments, a histamine level is measured. In some embodiments, the invention provides methods of detecting a mast cell activity biomarker of a mast-cell activity-associated disorder. In some embodiments, the method includes obtaining a sample from a human patient and detecting in the sample presence, level and/or location of one or more mast cell activity factors determined to be a mast cell activity biomarker of the mast cell activity-associated disorder. In some embodiments, the method comprises a step of comparing the presence, level and/or location of one or more mast cell activity factors with a presence, level and/or location of a mast cell activity factor.

### Biological Samples

Methods of the invention may be applied to any type of biological samples allowing one or more inventive biomarkers to be assayed. Examples of suitable biological samples include, but are not limited to, cerebrospinal fluid (CSF), lymphatic fluid, cells, tissue, whole blood, mouthwash, plasma, serum, urine, stool, saliva, cord blood, chorionic villus sample, chorionic villus sample culture, amniotic fluid, amniotic fluid culture, transcervical lavage fluid. Biological samples suitable for the inventive may be fresh or frozen samples collected from a subject, or archival samples with known diagnosis, treatment and/or outcome history. Biological samples may be collected by any invasive or non-invasive means, such as, for example, by drawing CSF or blood from a subject, or using fine needle aspiration or needle biopsy, or by surgical biopsy.

In certain embodiments, biological samples may be used without or with limited processing of the sample. For example, protein biomarkers may be prepared from a biological sample. In some embodiments, a protein extract contains the total protein content. In some embodiments, protein extracts containing one or more of membrane proteins, nuclear proteins, and cytosolic proteins may be prepared. For example, the cytosolic proteins may include the proteins present in the preformed granules of the mast cells. Methods of protein extraction are well known in the art (see, for example “Protein Methods”, D. M. Bollag et al., 2^{nd} Ed., 1996, Wiley-Liss; “Protein Purification Methods: A Practical Approach”, E. L. Harris and S. Angal (Eds.), 1989; “Protein Purification Techniques: A Practical Approach”, S. Roe, 2^{nd} Ed., 2001, Oxford University Press; “Principles and Reactions of Protein Extraction, Purification, and Characterization”, H. Ahmed, 2005, CRC Press: Boca Raton, Fla.). Numerous different and versatile kits can be used to extract proteins from bodily fluids and tissues, and are commercially available from, for example, BioRad Laboratories (Hercules, Calif.), BD Biosciences Clontech (Mountain View, Calif.), Chemicon International, Inc. (Temecula, Calif.), Calbiochem (San Diego, Calif.), Pierce Biotechnology (Rockford, Ill.), and Invitrogen Corp. (Carlsbad, Calif.). User Guides that describe in great detail the protocol to be followed are usually included in all these kits. Sensitivity, processing time and costs may be different from one kit to another. One of ordinary skill in the art can easily select the kit(s) most appropriate for a particular situation. After the protein extract has been obtained, the protein concentration of the extract is preferably standardized to a value being the same as that of the control sample in order to allow signals of the protein markers to be quantitated. Such standardization can be made using photometric or spectrometric methods or gel electrophoresis.

In some embodiments, nucleic acids may be extracted from a biological sample. For example, RNA may be extracted from the sample before analysis. In some embodiments, that RNA that is extracted is the mRNA expressed from the CYP21A2 gene. In some embodiments, RNA that is extracted is the mRNA expressed from the c-kit gene. Methods of RNA extraction are well known in the art (see, for example, J. Sambrook et al., “Molecular Cloning: A Laboratory Manual”, 1989, 2^{nd} Ed., Cold Spring Harbor Laboratory Press: Cold Spring Harbor, N.Y.). Most methods of RNA isolation from bodily fluids or tissues are based on the disruption of the tissue in the presence of protein denaturants to quickly and effectively inactivate RNases. Isolated total RNA may then be further purified from the protein contaminants and concentrated by selective ethanol precipitations, phenol/chloroform extractions followed by isopropanol precipitation or cesium chloride, lithium chloride or cesium trifluoroacetate gradient centrifugations. Kits are also available to extract RNA (i.e., total RNA or mRNA) from bodily fluids or tissues and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Qiagen, Inc. (Valencia, Calif.).

In certain embodiments, after extraction, mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2^{nd} Ed., Cold Spring Harbour Laboratory Press: New York; “Short Protocols in Molecular Biology”, F. M. Ausubel (Ed.), 2002, 5^{th} Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each probe being monitored, or using thermostable DNA polymerases (such as avian myeloblastosis virus reverse transcriptase or Moloney murine leukemia virus reverse transcriptase).

In some embodiments, lipid-derived biomarkers may be prepared from a biological sample. In some embodiments, a lipid extract contains the total lipid content. In some embodiments, lipid extracts containing one or more of membrane lipids, nuclear lipids, and cytosolic lipids may be prepared. For example, the cytosolic lipids may include the lipid derived metabolites present in the preformed granules of the mast cells.

### Methods of Detection

Measuring Protein Biomarkers

Measuring or detecting protein biomarker expression levels in a biological sample may be performed by any suitable method (see, for example, E. Harlow and A. Lane, “Antibodies: A Laboratories Manual”, 1988, Cold Spring Harbor Laboratory: Cold Spring Harbor, N.Y.).

In general, protein expression levels are determined by contacting a biological sample obtained from a subject with binding agents for one or more of protein biomarkers; detecting, in the sample, the levels of one or more protein biomarkers that bind to the binding agents; and comparing the levels of one or more protein biomarkers in the sample with the levels of the corresponding protein biomarkers in a control sample. As used herein, the term “binding agent” refers to an entity such as a polypeptide or antibody that specifically binds to an inventive protein biomarker. An entity “specifically binds” to a polypeptide if it reacts/interacts at a detectable level with the polypeptide but does not react/interact detectably with peptides containing unrelated sequences or sequences of different polypeptides.

In certain embodiments, a suitable binding agent is a ribosome, with or without a peptide component, an RNA molecule, or a polypeptide (e.g., a polypeptide that comprises a polypeptide sequence of a protein marker, a peptide variant thereof, or a non-peptide mimetic of such a sequence).

In other embodiments, a suitable binding agent is an antibody specific for a protein biomarker described herein (e.g., an antibody specific for any protein biomarker listed in Table 1). In some embodiments, a suitable antibody can specifically bind to a particular form of a protein biomarker, for example, a cytokine protein (e.g., IL-1, INF-γ)). Suitable antibodies for use in the methods of the present invention include monoclonal and polyclonal antibodies, immunologically active fragments (e.g., Fab or (Fab)₂ fragments), antibody heavy chains, humanized antibodies, antibody light chains, and chimeric antibodies. Antibodies, including monoclonal and polyclonal antibodies, fragments and chimeras, may be prepared using methods known in the art (see, for example, R. G. Mage and E. Lamoyi, in “Monoclonal Antibody Production Techniques and Applications”, 1987, Marcel Dekker, Inc.: New York, pp. 79-97; G. Kohler and C. Milstein, Nature, 1975, 256: 495-497; D. Kozbor et al., J. Immunol. Methods, 1985, 81: 31-42; and R. J. Cote et al., Proc. Natl. Acad. Sci. 1983, 80: 2026-203; R. A. Lerner, Nature, 1982, 299: 593-596; A. C. Nairn et al., Nature, 1982, 299: 734-736; A. J. Czernik et al., Methods Enzymol. 1991, 201: 264-283; A. J. Czernik et al., Neuromethods: Regulatory Protein Modification: Techniques & Protocols, 1997, 30: 219-250; A. J. Czernik et al., Neuroprotocols, 1995, 6: 56-61; H. Zhang et al., J. Biol. Chem. 2002, 277: 39379-39387; S. L. Morrison et al., Proc. Natl. Acad. Sci., 1984, 81: 6851-6855; M. S. Neuberger et al., Nature, 1984, 312: 604-608; S. Takeda et al., Nature, 1985, 314: 452-454). Antibodies to be used in the methods of the invention can be purified by methods well known in the art (see, for example, S. A. Minden, “Monoclonal Antibody Purification”, 1996, IBC Biomedical Library Series: Southbridge, Mass.). For example, antibodies can be affinity-purified by passage over a column to which a protein marker or fragment thereof is bound. The bound antibodies can then be eluted from the column using a buffer with a high salt concentration.

Instead of being prepared, antibodies to be used in the methods of the present invention may be obtained from scientific or commercial sources (e.g., Cayman Chemical).

Labeled Binding Agents.

In certain embodiments, the binding agent is directly or indirectly labeled with a detectable moiety. The role of a detectable agent is to facilitate the detection step of the diagnostic method by allowing visualization of the complex formed by binding of the binding agent to the protein marker (or analog or fragment thereof). Preferably, the detectable agent is selected such that it generates a signal which can be measured and whose intensity is related (preferably proportional) to the amount of protein marker present in the sample being analyzed. Methods for labeling biological molecules such as polypeptides and antibodies are well-known in the art (see, for example, “Affinity Techniques. Enzyme Purification: Part B”, Methods in Enzymol., 1974, Vol. 34, W. B. Jakoby and M. Wilneck (Eds.), Academic Press: New York, N.Y.; and M. Wilchek and E. A. Bayer, Anal. Biochem., 1988, 171: 1-32).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available. In some embodiments, a protein mast cell activity biomarker is detected by contacting the subject or sample with a molecular contrast agent to visualize mast cell degranulation.

In certain embodiments, the binding agents (e.g., antibodies) may be immobilized on a carrier or support (e.g., a bead, a magnetic particle, a latex particle, a microtiter plate well, a cuvette, or other reaction vessel). Examples of suitable carrier or support materials include agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, and the like. Binding agents may be indirectly immobilized using second binding agents specific for the first binding agents (e.g., mouse antibodies specific for the protein markers may be immobilized using sheep anti-mouse IgG Fc fragment specific antibody coated on the carrier or support).

Protein expression levels in a biological sample may be determined using immunoassays. Examples of such assays are time resolved fluorescence immunoassays (TR-FIA), radioimmunoassays, enzyme immunoassays (e.g., ELISA), immunofluorescence immunoprecipitation, latex agglutination, hemagglutination, Western blot, and histochemical tests, which are conventional methods well-known in the art. As will be appreciated by one skilled in the art, the immunoassay may be competitive or non-competitive. Methods of detection and quantification of the signal generated by the complex formed by binding of the binding agent with the protein marker will depend on the nature of the assay and of the detectable moiety (e.g., fluorescent moiety).

Alternatively, the protein biomarker expression levels may be determined using mass spectrometry based methods or image (including use of labeled ligand) based methods known in the art for the detection of proteins. Other suitable methods include 2D-gel electrophoresis, proteomics-based methods. Proteomics, which studies the global changes of protein expression in a sample, can include the following steps: (1) separation of individual proteins in a sample by electrophoresis (1-D PAGE), (2) identification of individual proteins recovered from the gel (e.g., by mass spectrometry or N-terminal sequencing), and (3) analysis of the data using bioinformatics.

Measuring Nucleic Acid Biomarkers

Measuring or detection of expression levels of nucleic acids in a biological sample may be performed by any suitable method, including, but not limited to, hybridization (e.g., Southern or Northern analysis), polymerase chain reaction (PCR) (see, for example, U.S. Pat. Nos. 4,683,195; 4,683,202, and 6,040,166; “PCR Protocols: A Guide to Methods and Applications”, Innis et al. (Eds.), 1990, Academic Press: New York), reverse transcriptase PCR (RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320 308), one-sided PCR (Ohara et al., Proc. Natl. Acad. Sci., 1989, 86: 5673-5677), in situ hybridization, Taqman-based assays (Holland et al., Proc. Natl. Acad. Sci., 1991, 88: 7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan™, and the like.

Nucleic acid probes for use in the detection of polynucleotide sequences in biological samples may be constructed using conventional methods known in the art. Suitable probes may be based on nucleic acid sequences encoding at least 5 sequential amino acids from regions of nucleic acids encoding a biomarker, and preferably comprise about 15 to about 50 nucleotides. A nucleic acid probe may be labeled with a detectable moiety, as mentioned above in the case of binding agents. The association between the nucleic acid probe and detectable moiety can be covalent or non-covalent. Detectable moieties can be attached directly to nucleic acid probes or indirectly through a linker (E. S. Mansfield et al., Mol. Cell. Probes, 1995, 9: 145-156). Methods for labeling nucleic acid molecules are well-known in the art (for a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153).

Nucleic acid probes may be used in hybridization techniques to detect polynucleotides encoding biomarkers. The technique generally involves contacting and incubating nucleic acid molecules in a biological sample obtained from a subject with the nucleic acid probes under conditions such that specific hybridization takes place between the nucleic acid probes and the complementary sequences in the nucleic acid molecules. Typically, stringent hybridization conditions are used. In some embodiments, “stringent hybridization conditions” refer to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In some embodiments, stringent hybridization conditions should not allow for hybridization of two nucleic acids which differ over a stretch of 20 contiguous nucleotides by more than two bases. After incubation, the non-hybridized nucleic acids are removed, and the presence and amount of nucleic acids that have hybridized to the probes are detected and quantified.

Detection of nucleic acid molecules comprising polynucleotide sequences coding for a biomarker may involve amplification of specific polynucleotide sequences using an amplification method such as PCR (e.g., RT-PCR), followed by analysis of the amplified molecules using techniques known in the art. Suitable primers can be routinely designed by one skilled in the art. In order to maximize hybridization under assay conditions, primers and probes employed in the methods of the invention generally have at least 60%, preferably at least 75% and more preferably at least 90% identity to a portion of nucleic acids encoding a biomarker.

Hybridization and amplification techniques described herein may be used to assay qualitative and quantitative aspects of expression of nucleic acid molecules comprising polynucleotide sequences coding for inventive biomarkers described herein.

Alternatively, oligonucleotides or longer fragments derived from nucleic acids encoding each biomarker may be used as targets in a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state level of large numbers of polynucleotide sequences simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; J. J. Chen et al., Genomics, 1998, 51: 313-324; U.S. Pat. Nos. 5,143,854; 5,445,934; 5,807,522; 5,837,832; 6,040,138; 6,045,996; 6,284,460; and 6,607,885).

Measuring Lipid-Derived Biomarkers

Lipid-derived biomarkers may be readily isolated and/or quantified by methods known to those of skill in the art, including, but not limited to, methods utilizing: mass spectrometry (MS), high performance liquid chromatography (HPLC), isocratic HPLC, gradient HPLC, normal phase chromatography, reverse phase HPLC, size exclusion chromatography, ion exchange chromatography, capillary electrophoresis, microfluidics, chromatography, gas chromatography (GC), thin-layer chromatography (TLC), immobilized metal ion affinity chromatography (IMAC), affinity chromatography, immunoassays, and/or colorimetric assays. In some embodiments, the methods of the invention utilize MS to determine lipid-derived biomarker presence. In some embodiments, the methods of the invention utilize an immunoassay to determine lipid-derived biomarker presence. In one embodiment, the methods of the invention utilize MS to determine the level of a biomarker. In one embodiment, the methods of the invention utilize an immunoassay to determine the level of a biomarker. In some embodiments, a lipid-derived mast cell activity biomarker is detected by contacting the subject or sample with a molecular contrast agent to visualize mast cell degranulation.

Measuring Histamine

Histamine, and its metabolites N-methylhistamine, may be readily isolated and/or quantified by methods known to those of skill in the art, including, but not limited to, methods utilizing: using fluorimetric and colorimetric techniques that measure its extracellular presence, level and/or location. Histamine and its metabolites may be detected and quantified using high performance liquid chromatography (HPLC), thin layer chromatography (TLC), fluorometric glass fiber-based histamine method, or by radioimmunoassay (Granderus et al., Agents Actions (1984) 14:341-345; Andersson el al., J. Allergy Clin. Immunol. (1990) 86:815-820). In some embodiments, histamine is detected by contacting the subject or sample with a molecular contrast agent to visualize mast cell degranulation.

Methods of Analysis of Allelic Variants

Methods of the present invention include methods of analyzing a gene for allelic variation to determine whether the allelic variation correlates with the presence, susceptibility to, or severity of a mast cell activity-associated disorder.

The source of the gene, or a polynucleotide, is typically a biological sample that includes genomic DNA and/or RNA. A biological sample may be treated to obtain DNA or RNA for analysis.

Methods of analysis of polynucleotides are widely known in the art and include, in some aspects, amplification of the polynucleotide to form amplified polynucleotides, preferably including amplified nucleotides that correspond to an exon from a gene of interest (e.g., CYP21A2), and detecting the amplified polynucleotides. Preferably, nucleotides are amplified by PCR. The conditions for amplifying a polynucleotide by PCR vary depending on the nucleotide sequence of primers used, and methods for determining such conditions are routine in the art.

Various types of amplification techniques are known and used routinely, such as allele-specific PCR, cold PCR, hot PCR, reverse-transcriptase PCR, and the like. These and other amplification techniques are known in the art and are used routinely. In view of the disclosure of SEQ ID NO:1, the skilled person can easily adapt an amplification technique to be used in identifying mutations in a CYP21A2 polynucleotide.

After amplification, the sizes of the amplified polynucleotides may be determined, for instance by gel electrophoresis, and compared. The amplified polynucleotides can be visualized by staining (e.g., with ethidium bromide) or labeling with a suitable label known to those skilled in the art, including radioactive and nonradioactive labels. Typical radioactive labels include ³³P. Nonradioactive labels include, for example, ligands such as biotin or digoxigenin as well as enzymes such as phosphatase or peroxidases, or the various chemiluminescers such as luciferin, or fluorescent compounds like fluorescein and its derivatives. Optionally, the nucleotide sequence of an amplified polynucleotide can be determined.

In another aspect of the methods for analyzing a polynucleotide containing a mutation, or allelic variant polynucleotide probes are used that hybridize to a polynucleotide. As used herein, “hybridizes,” “hybridizing,” and “hybridization” means that a probe forms a noncovalent interaction with a target polynucleotide under standard conditions. Standard hybridizing conditions are those conditions that allow a probe to hybridize to a target polynucleotide. Such conditions are readily determined for a probe and the target polynucleotide using techniques well known to the art, for example see Sambrook et al. Molecular Cloning: A Laboratory Manual; Cold Spring Harbor Laboratory: New York (1989).

In one embodiment of this aspect of the invention, the methods include digesting genomic DNA of a subject with a restriction endonuclease to obtain polynucleotides, and probing the polynucleotides under hybridizing conditions with a detectably labeled probe. The digestion of genomic DNA with endonucleases is routine in the art, and numerous endonucleases are known. Typically, the polynucleotides resulting from digestion are fractionated, for instance by gel electrophoresis, denatured to yield single stranded polynucleotides, and then exposed to the probe under hybridizing conditions. The probe that has hybridized to the polynucleotide is then detected, and the size of the hybridized polynucleotide may then be determined. The presence or absence of the mutation can be inferred by the approximate molecular weight of the detected polynucleotide. The presence of a mutation indicates the person has or is at risk, and the absence of a mutation indicates the person is not at risk.

Other methods can be used to analyze a polynucleotide. Examples include, but are not limited to, ligase-mediated detection techniques, fluorescent in situ hybridization, direct DNA sequencing, PFGE analysis, Southern or Northern blotting, single-stranded conformation analysis (SSCA), RNAse protection assay, allele-specific oligonucleotide, dot blot analysis, denaturing gradient gel electrophoresis, RFLP, PCR-SSCP and next generation sequencing.

Comparing to a Reference

In some embodiments of the present invention, once a presence or expression level of a mast cell activity factor and/or biomarker (e.g., biomarkers selected from Table 1) has been determined (as described above) for a biological sample being measured, presence and/or expression level can be compared to presence and/or expression level in one or more reference or control samples. In some embodiments, comparison of presence and/or expression levels according to methods of the present invention is preferably performed after the presence and/or expression levels obtained have been corrected for both differences in the amount of sample assayed and variability in the quality of the sample used (e.g., amount of protein or lipid-derived metabolite extracted, or amount and quality of mRNA tested). In some embodiments, correction may be carried out using different methods well-known in the art. For example, the protein concentration of a sample may be standardized using photometric or spectrometric methods or gel electrophoresis before the sample is analyzed. In some embodiments, the lipid-derived metabolite concentration of a sample may be standardized using an internal standard which is added to the sample before it is analyzed. In some embodiments, samples containing nucleic acid molecules may be corrected by normalizing the levels against reference genes (e.g., housekeeping genes) in the same sample. Alternatively or additionally, normalization can be based on the mean or median signal (e.g., Ct in the case of RT-PCR) of all assayed genes or a large subset thereof (global normalization approach).

In some embodiments, a reference or control sample is derived from a healthy subject (e.g., a subject not suffering from the mast cell activity-associated disorder). In some embodiments, a reference sample is derived from a group of healthy subjects. In some embodiments, a reference sample is derived from the subject suffering from the mast cell activity-associated disorder prior to onset of the disorder. In some embodiments, a reference sample is a historical sample. In some embodiments, a level of a mast cell activity factor and/or biomarker in the biological sample represents a level that is more than about 20%, 30%, 40%, 50%, 60%, 70%, 80%, 90%, 95%, 98%, 1-fold, 1.2-fold, 1.5-fold, 1.75-fold, 2-fold, 2.25-fold, 2.5-fold, 2.75-fold, 3-fold, 4-fold, 5-fold, 10-fold, 50-fold, 100-fold or 500-fold higher or lower than the level of the mast cell activity marker or biomarker in the reference sample. In some embodiments, a level of a mast cell activity factor and/or biomarker in a biological sample represents a level that is about 20% to about 98%, about 20% to about 90%, about 20% to about 80%, about 20% to about 70%, about 20% to about 60%, about 20% to about 50%, about 30% to about 98%, about 30% to about 30%, about 30% to about 80%, about 30% to about 70%, about 30% to about 60%, about 30% to about 50%, about 40% to about 98%, about 40% to about 90%, about 40% to about 80%, about 40% to about 70%, about 40% to about 60% or about 40% to about 50% higher or lower than the level of the mast cell activity marker or biomarker in the reference sample. In some embodiments, a level of a mast cell activity factor and/or biomarker in a biological sample represents a level that is about 2-fold to about 500-fold, 2-fold to about 100-fold, 2-fold to about 50-fold, 2-fold to about 10-fold or 2-fold to about 5-fold higher or lower than the level of the mast cell activity marker or biomarker in the reference sample.

### Methods of Treating Mast Cell Activity-Associated Disorders

The present disclosure encompasses the insight that determination of factors and biomarkers associated with mast cell activity-associated disorders (e.g., MS, susceptibility to MS relapse, autoimmune disorders, neuroinflammatory disorders, cancer) is useful for identification and/or administration of specific therapies for mast cell activity associated diseases, disorders and/or conditions as described herein. For example, having learned of or determined that a particular disease, disorder and/or condition is a mast cell activity associated disease, disorder and/or condition, and/or is characterized by one or more features whose presence, level and/or location correlates with a mast cell activity biomarker as described herein, those skilled in the art will be aware of therapeutic modalities targeting relevant mast cell activity that can be administered to treat the disease, disorder and/or condition.

Among other things, the present disclosure provides methods of treatment comprising administering mast cell inhibition therapy to a subject when a mast cell activity biomarker is detected in a sample from the subject. In some embodiments, mast cell inhibition therapy is administered in combination with one or more other therapies for treatment of the disease, disorder and/or condition.

In particular embodiments, the present disclosure provides compositions and/or methods of treating MS, MS relapse and/or another neuroinflammatory disease, disorder and/or condition in a subject, for example by administration of mast cell inhibition therapy (e.g., administration of a mast cell activity inhibitor) as described herein, when the presence of a mast cell activity biomarker has been detected in a sample from the subject. In some embodiments, a subject is diagnosed as suffering from or susceptible to the mast cell activity-associated disorder. In some embodiments, the present disclosure defines patient population(s) sensitive to treatment with a mast cell activity inhibitor as described herein. In some embodiments, the present disclosure provides methods of treatment by administration of therapy to such defined patient population(s).

Without wishing to be bound by any particular theory, mast cell activity inhibitors can include various classes of molecules that are therapeutically effective for the treatment of a mast cell activity-associated disease, disorder and/or condition. For example, a mast cell activity inhibitor may include a T-cell receptor ligand that competes with or inhibits one or more T-cell activating disorders. In some embodiments, a mast cell activity inhibitor is or comprises a mast cell adhesion inhibitor, an inhibitor of mast cell degranulation products and/or a B-cell activation inhibitor. In some embodiments, an inhibitor of mast cell degranulation products is hydroxyzine, a histamine receptor-1 antagonist, that has been shown to inhibit the progression and severity of EAE, and degranulation of mast cells within the thalamus (Dimitriadou et al., Int. J. Immunopharmacol. (2000) 22:673-684). A small open label study of hydroxyzine in 20 MS patients showed stability or improvement neurologically (Logothetis et al., Int. J. Immunopath. Pharma col. (2005) 18:771-778). In some embodiments, a mast cell activity inhibitor is or comprises a gene therapy agent that corrects mutations in the cytochrome P450 family, and in particular, allelic variations in the CYP21A2 gene.

In some embodiments, a mast cell activity inhibitor is an inhibitor of survival, migration and/or activity of mast cells. For example, masitinib is a selective oral tyrosine kinase inhibitor, that effectively inhibits the survival, migration and activity of mast cells and has shown some efficacy in a phase II trial of 35 progressive MS patients, (Vermersch et al., BMC Neurol. (2012) 12:36). In some embodiments, a mast cell activity inhibitor is Imatinib (i.e., Gleevac), a tyrosine kinase inhibitor that exerts anti-proliferative activity and immunomodulatory effects in lymphocytes, macrophages, mast cells and dendritic cells by abrogating multiple signal transduction pathways involved in pathogenesis of autoimmune diseases e.g., inhibiting the pro-inflammatory cytokines IFN-γ, TNF-α, IL-1β and IL-17 and MMPs secretion. In some embodiments, a mast cell activity inhibitor is a MS disease modifying treatments (DMTs), for example, Natalizumab may affect mast cells (Kritas et al., Int. J. Immunopathol. Pharmacol. (2014) 27:331-335), and dimethyl fumarate induces apoptosis of mast cells in vitro (Forster et al., Exp. Dermatol. (2013) 22:719-724.

Those of ordinary skill in the art will appreciate that appropriate formulations, indications, and dosing regimens are typically analyzed and approved by government regulatory authorities such as the Food and Drug Administration in the United States. In many embodiments, a mast cell activity inhibitor is administered in accordance with the present invention according to such an approved protocol. However, the present disclosure provides certain technologies for identifying, characterizing, and/or selecting particular patients to whom a mast cell activity inhibitor may desirably be administered. In some embodiments, insights provided by the present disclosure permit dosing of a given mast cell activity inhibitor with greater frequency and/or greater individual doses (e.g., due to reduced susceptibility to and/or incidence or intensity of undesirable effects) relative to that recommended or approved based on population studies that include both individuals identified as described herein (e.g., expressing mast cell activity factors and/or biomarkers) and other individuals. In some embodiments, insights provided by the present disclosure permit dosing of a given mast cell activity inhibitor with reduced frequency and/or reduced individual doses (e.g., due to increased responsiveness) relative to that recommended or approved based on population studies that include both individuals identified as described herein (e.g., expressing mast cell activity factors and/or biomarkers) and other individuals.

In some embodiments, a mast cell activity inhibitor is administered in a pharmaceutical composition that also comprises a physiologically acceptable carrier or excipient. In some embodiments, a pharmaceutical composition is sterile. In many embodiments, a pharmaceutical composition is formulated for a particular mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrrolidone, etc., as well as combinations thereof. A pharmaceutical preparation can, if desired, comprise one or more auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. In some embodiments, a water-soluble carrier suitable for intravenous administration is used.

In some embodiments, a pharmaceutical composition or medicament, if desired, can contain an amount (typically a minor amount) of wetting or emulsifying agents, and/or of pH buffering agents. In some embodiments, a pharmaceutical composition can be a liquid solution, suspension, emulsion, tablet, pill, capsule, sustained release formulation, or powder. In some embodiments, a pharmaceutical composition can be formulated as a suppository, with traditional binders and carriers such as triglycerides. Oral formulation can include standard carriers such as pharmaceutical grades of mannitol, lactose, starch, magnesium stearate, polyvinyl pyrrolidone, sodium saccharine, cellulose, magnesium carbonate, etc.

In some embodiments, a pharmaceutical composition can be formulated in accordance with the routine procedures as a pharmaceutical composition adapted for administration to human beings. For example, in some embodiments, a composition for intravenous administration typically is a solution in sterile isotonic aqueous buffer. Where necessary, a composition may also include a solubilizing agent and a local anesthetic to ease pain at the site of the injection. Generally, ingredients are supplied either separately or mixed together in unit dosage form, for example, as a dry lyophilized powder or water free concentrate in a hermetically sealed container such as an ampule or sachet indicating the quantity of active agent. Where a composition is to be administered by infusion, it can be dispensed with an infusion bottle containing sterile pharmaceutical grade water, saline or dextrose/water. Where a composition is administered by injection, an ampule of sterile water for injection or saline can be provided so that the ingredients may be mixed prior to administration.

In some embodiments, a mast cell activity inhibitor can be formulated in a neutral form. In some embodiments, it may be formulated in a salt form. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Pharmaceutical compositions for use in accordance with the present invention may be administered by any appropriate route. In some embodiments, a pharmaceutical composition is administered intravenously. In some embodiments, a pharmaceutical composition is administered subcutaneously. In some embodiments, a pharmaceutical composition is administered by direct administration to a target tissue, such as heart or muscle (e.g., intramuscular), or nervous system (e.g., direct injection into the brain; intraventricularly; intrathecally). Alternatively or additionally, in some embodiments, a pharmaceutical composition is administered parenterally, transdermally, or transmucosally (e.g., orally or nasally). More than one route can be used concurrently, if desired. In some embodiments of the invention, a mast cell activity inhibitor is delivered to a subject by a nanobot. For example, the method of treating a mast cell activity-associated disorder in a subject comprises administering a nanobot agent adapted to detect presence of a mast cell activity biomarker and to deliver a mast cell activity inhibitor, so that the mast cell activity inhibitor is administered when the biomarker is detected. In some embodiments, a mast cell activity inhibitor modulates mast cell activity and is delivered by a nanobot agent prior to, during or after degranulation or activation of a mast cell. In some embodiments, a mast cell activity inhibitor neutralizes mast cell metabolites released during or after degranulation and is delivered by a nanobot agent. In some embodiments, a mast cell activity modulator is delivered in conjunction with, prior to, or after delivery of a mast cell metabolite neutralizer.

A mast cell activity inhibitor (or a composition or medicament containing a mast cell activity inhibitor, can be administered alone, or in conjunction with other mast cell activity inhibitors when the presence of a mast cell activity biomarker has been detected in a sample from the subject. The term, “in conjunction with,” indicates that a first mast cell activity inhibitor is administered prior to, at about the same time as, or following another mast cell activity inhibitor. For example, a first mast cell activity inhibitor can be mixed into a composition containing one or more different mast cell activity inhibitors, and thereby administered contemporaneously; alternatively, the agent can be administered contemporaneously, without mixing (e.g., by “piggybacking” delivery of the agent on the intravenous line by which the second mast cell activity inhibitor is also administered, or vice versa). In another example, the mast cell activity inhibitor can be administered separately (e.g., not admixed), but within a short time frame (e.g., within 24 hours) of administration of the second mast cell activity inhibitor.

In some embodiments of the invention, a first agent is selected from the group consisting of chondroitin, methylsulfonylmethane (MSM), glucosamine, an H₁ receptor antagonist, an H₂ receptor antagonist, and combinations thereof. In some embodiments, a second agent is selected from the group consisting of selective serotonin reuptake inhibitors (SSRIs), norepinephrine-dopamine reuptake inhibitors (NDRIs), and combinations thereof.

In some embodiments, subjects treated with mast cell activity inhibitors are administered one or more immunosuppressants. In some embodiments, one or more immunosuppressants are administered to decrease, inhibit, or prevent an undesired autoimmune response (e.g., enterocolitis, hepatitis, dermatitis (including toxic epidermal necrolysis), neuropathy, and/or endocrinopathy), for example, hypothyroidism. Exemplary immunosuppressants include steroids, antibodies, immunoglobulin fusion proteins, and the like. In some embodiments, an immunosuppressant inhibits B cell activity (e.g., rituximab). In some embodiments, an immunosuppressant is a decoy polypeptide antigen.

In some embodiments, mast cell activity inhibitors (or a composition or medicament containing mast cell activity inhibitors) are administered in a therapeutically effective amount (e.g., a dosage amount and/or according to a dosage regimen that has been shown, when administered to a relevant population, to be sufficient to treat a mast cell activity-associated disorder, such as by ameliorating symptoms associated with the mast cell activity-associated disorder, preventing or delaying the onset of the mast cell activity-associated disorder, and/or lessening the severity or frequency of symptoms of mast cell activity-associated disorder, predicting, preventing or treating MS relapse). In some embodiments, long term clinical benefit is observed after treatment with mast cell activity inhibitors. Those of ordinary skill in the art will appreciate that a dose which will be therapeutically effective for the treatment of a mast cell activity-associated disorder in a given patient may depend, at least to some extent, on the nature and extent of mast cell activity-associated disorder, and can be determined by standard clinical techniques. In some embodiments, one or more in vitro or in vivo assays may optionally be employed to help identify optimal dosage ranges. In some embodiments, a particular dose to be employed in the treatment of a given individual may depend on the route of administration, the extent of the mast cell activity-associated disorder, and/or one or more other factors deemed relevant in the judgment of a practitioner in light of patient's circumstances. In some embodiments, effective doses may be extrapolated from dose-response curves derived from in vitro or animal model test systems (e.g., as described by the U.S. Department of Health and Human Services, Food and Drug Administration, and Center for Drug Evaluation and Research in “Guidance for Industry: Estimating Maximum Safe Starting Dose in Initial Clinical Trials for Therapeutics in Adult Healthy Volunteers”, Pharmacology and Toxicology, July 2005.

In some embodiments, a therapeutically effective amount of a mast cell activity inhibitor can be, for example, more than about 0.01 mg/kg, more than about 0.05 mg/kg, more than about 0.1 mg/kg, more than about 0.5 mg/kg, more than about 1.0 mg/kg, more than about 1.5 mg/kg, more than about 2.0 mg/kg, more than about 2.5 mg/kg, more than about 5.0 mg/kg, more than about 7.5 mg/kg, more than about 10 mg/kg, more than about 12.5 mg/kg, more than about 15 mg/kg, more than about 17.5 mg/kg, more than about 20 mg/kg, more than about 22.5 mg/kg, or more than about 25 mg/kg body weight. In some embodiments, a therapeutically effective amount of a mast cell activity inhibitor can be about 0.01-25 mg/kg, about 0.01-20 mg/kg, about 0.01-15 mg/kg, about 0.01-10 mg/kg, about 0.01-7.5 mg/kg, about 0.01-5 mg/kg, about 0.01-4 mg/kg, about 0.01-3 mg/kg, about 0.01-2 mg/kg, about 0.01-1.5 mg/kg, about 0.01-1.0 mg/kg, about 0.01-0.5 mg/kg, about 0.01-0.1 mg/kg, about 1-20 mg/kg, about 4-20 mg/kg, about 5-15 mg/kg, about 5-10 mg/kg body weight. In some embodiments, a therapeutically effective amount of a mast cell activity inhibitor is about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg about 1.4 mg/kg, about 1.5 mg/kg, about 1.6 mg/kg, about 1.7 mg/kg, about 1.8 mg/kg, about 1.9 mg/kg, about 2.0 mg/kg, about 2.5 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, body weight, or more. In some embodiments, the therapeutically effective amount of a mast cell activity inhibitor is no greater than about 30 mg/kg, no greater than about 20 mg/kg, no greater than about 15 mg/kg, no greater than about 10 mg/kg, no greater than about 7.5 mg/kg, no greater than about 5 mg/kg, no greater than about 4 mg/kg, no greater than about 3 mg/kg, no greater than about 2 mg/kg, or no greater than about 1 mg/kg body weight or less.

In some embodiments, an administered dose for a particular individual is varied (e.g., increased or decreased) over time, depending on the needs of the individual.

In yet another example, a loading dose (e.g., an initial higher dose) of a therapeutic composition may be given at the beginning of a course of treatment, followed by administration of a decreased maintenance dose (e.g., a subsequent lower dose) of the therapeutic composition.

It will be appreciated that a loading dose and maintenance dose amounts, intervals, and duration of treatment may be determined by any available method, such as those exemplified herein and those known in the art. In some embodiments, a loading dose amount of a mast cell activity inhibitor is about 0.01-1 mg/kg, about 0.01-5 mg/kg, about 0.01-10 mg/kg, about 0.1-10 mg/kg, about 0.1-20 mg/kg, about 0.1-25 mg/kg, about 0.1-30 mg/kg, about 0.1-5 mg/kg, about 0.1-2 mg/kg, about 0.1-1 mg/kg, or about 0.1-0.5 mg/kg body weight. In some embodiments, a maintenance dose amount of a mast cell activity inhibitor is about 0-10 mg/kg, about 0-5 mg/kg, about 0-2 mg/kg, about 0-1 mg/kg, about 0-0.5 mg/kg, about 0-0.4 mg/kg, about 0-0.3 mg/kg, about 0-0.2 mg/kg, about 0-0.1 mg/kg body weight. In some embodiments, a loading dose of a mast cell activity inhibitor is administered to an individual at regular intervals for a given period of time (e.g., 1, 2, 3, 4, 5, 6, 7, 8, 9, 10, 11, 12 or more months) and/or a given number of doses (e.g., 1, 2, 3, 4, 5, 6, 7, 8, 9, 10, 15, 20, 25, 30 or more doses), followed by maintenance dosing. In some embodiments, a maintenance dose of a mast cell activity inhibitor ranges from 0-2 mg/kg, about 0-1.5 mg/kg, about 0-1.0 mg/kg, about 0-0.75 mg/kg, about 0-0.5 mg/kg, about 0-0.4 mg/kg, about 0-0.3 mg/kg, about 0-0.2 mg/kg, or about 0-0.1 mg/kg body weight. In some embodiments, a maintenance dose of a mast cell activity inhibitor is about 0.01, 0.02, 0.04, 0.06, 0.08, 0.1, 0.2, 0.3, 0.4, 0.5, 0.6, 0.7, 0.8, 0.9, 1.0, 1.2, 1.4, 1.6, 1.8, or 2.0 mg/kg body weight. In some embodiments, a maintenance dose of a mast cell activity inhibitor is administered for 1, 2, 3, 4, 5, 6, 7, 8, 9, 10, 11, 12 or more months. In some embodiments, maintenance dosing is administered for 1, 2, 3, 4, 5, 6, 7, 8, 9, 10 or more years. In some embodiments, maintenance dosing is administered indefinitely (e.g., for life time).

A therapeutically effective amount of a mast cell activity inhibitor may be administered as a one-time dose or administered at intervals, depending on the nature and extent of the mast cell activity-associated disorder, and on an ongoing basis. Administration at an “interval,” as used herein indicates that the therapeutically effective amount is administered periodically (as distinguished from a one-time dose). The interval can be determined by standard clinical techniques. In some embodiments, a mast cell activity inhibitor is administered bimonthly, monthly, twice monthly, triweekly, biweekly, weekly, twice weekly, thrice weekly, or daily. The administration interval for a single individual need not be a fixed interval, but can be varied over time, depending on the needs and rate of recovery of the individual.

As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day.

The invention additionally pertains to a pharmaceutical composition comprising a mast cell activity inhibitor, as described herein, in a container (e.g., a vial, bottle, bag for intravenous administration, syringe, etc.) with a label containing instructions for administration of the composition for treatment of a mast cell activity-associated disorder.

### Methods of Diagnosis of Mast Cell Activity-Associated Disorders

It is contemplated that one or more mast cell activity factors and/or biomarkers whose expression profiles correlate with a mast cell activity-associated disorder can diagnose the disorder, discriminate between different stages of the disorder, determine the severity of the disorder, and/or assess risk for developing the disorder. Accordingly, in some embodiments, the present invention provides methods for analyzing biological samples obtained from a subject suspected of having a mast cell activity-associated disorder to measure expression levels of biomarkers described herein, to determine if the subject has a mast cell activity-associated disorder, is at risk of developing a mast cell activity-associated disorder, or to determine the severity of the mast cell activity-associated disorder. In some embodiments, the present invention provides methods of diagnosing a subject as susceptible to MS relapse, the method comprising detected a presence, level and/or location of a mast cell activity biomarker in the subject, or in a sample from the subject, and predicting the occurrence of MS relapse based on the mast cell activity biomarker that is detected. In some embodiments, a mast cell activity inhibitor, as described herein, is administered to treat the MS relapse.

Typically, in such methods, the biomarkers' levels determined or measured for a biological sample obtained from the subject are compared to one or more control levels. Various control levels of the biomarkers may be used. For example, suitable control levels may be indicative of the levels of the one or more biomarkers in a control subject who does not have the mast cell activity-associated disorder. Such control levels may be obtained by measuring the corresponding one or more mast cell activity factors and/or biomarkers simultaneously under the same conditions in a control sample obtained from one or more healthy control subjects. Suitable control samples may be obtained from one healthy control individual or pooled from a plurality of healthy control individuals. In some embodiments, a control level indicative of the level of a factor or biomarker in healthy individuals can be determined from a significant number of individuals, and an average or mean is obtained. Typically, a healthy control individual is at a comparable age or other development state. In some embodiments, a suitable control level for a factor or biomarker is a numerical reference based on historical data, also referred to as a historical control (i.e., of a test or assay performed previously, or an amount or result that is previously known). In some embodiments, a control level is or comprises a printed or otherwise saved record. In some embodiments, an elevated level with statistical significance of the one or more biomarkers as compared to a suitable control level indicates that the subject has a mast cell activity-associated disorder, is at risk of developing a mast cell activity-associated disorder. In some embodiments, a diminished level with statistical significance of the one or more biomarkers as compared to a suitable control level indicates that the subject has a mast cell activity-associated disorder, or is at risk of developing a mast cell activity-associated disorder. Various statistical techniques and analysis methods may be used to determine if a biomarker has an elevated or diminished level with statistical significance (i.e., the difference is not caused by random variations). Exemplary statistical techniques and methods include, but are not limited to, Linear and Quadradic discriminant analysis, k-nearest neighbor. In some embodiments, a factor or biomarker has an elevated level if the level of the factor or biomarker measured in biological samples is more than 20%, 30%, 40%, 50%, 60%, 70%, 80%, 90%, 1-fold, 1.2-fold, 1.5-fold, 1.75-fold, 2-fold, 2.25-fold, 2.5-fold, 2.75-fold, or 3-fold higher as compared to a control level. In some embodiments, a factor or biomarker has a diminished level if the level of the factor or biomarker measured in biological samples is reduced by more than 10%, 20%, 30%, 40%, 50%, 60%, 70%, 80%, 90%, or 95% as compared to a control level.

In some embodiments, control levels indicative of the levels of the corresponding one or more factors or biomarkers in a control subject who is suffering from a mast cell activity-associated disorder may be used. Such control levels may be determined by measuring the corresponding one or more factors or biomarkers simultaneously under the same conditions in control samples obtained from a control individual or pooled from a plurality of control individuals. In some embodiments, a control level indicative of the level of a factor or biomarker in individuals suffering from a mast cell activity-associated disorder can be determined from a significant number of individuals, and an average or mean is obtained. Typically, a suitable control individual is suffering from the same type of mast cell activity-associated disorder and at a substantially the same disease and developmental stage (e.g., same age and with similar disease symptoms). In some embodiments, a suitable control level may be a numerical threshold established based on historical data (i.e., of a test or assay performed previously, or an amount or result that is previously known) that correlates with a subject who has a mast cell activity-associated disorder, is at risk of developing a mast cell activity-associated disorder, or is a carrier of a mast cell activity-associated disorder. In these embodiments, a substantially similar level within statistic error margin or, an elevated or diminished level with statistical significance, of the one or more factors or biomarkers measured in a biological sample as compared to a suitable control level indicates that the subject has a mast cell activity-associated disorder, or is at risk of developing a mast cell activity-associated disorder. Various statistical methods and techniques such as those described herein may be used to determine statistical error margin and statistical significance. In some embodiments, a factor or biomarker has an elevated level if the level of the factor or biomarker measured in biological samples is more than 20%, 30%, 40%, 50%, 60%, 70%, 80%, 90%, 1-fold, 1.2-fold, 1.5-fold, 1.75-fold, 2-fold, 2.25-fold, 2.5-fold, 2.75-fold, or 3-fold higher as compared to a control level. In some embodiments, a factor or a biomarker has a diminished level if the level of the factor or biomarker measured in biological samples is reduced by more than 10%, 20%, 30%, 40%, 50%, 60%, 70%, 80%, 90%, or 95%, 1-fold, 1.2-fold, 1.5-fold, 1.75-fold, 2-fold, 2.25-fold, 2.5-fold, 2.75-fold, or 3-fold as compared to a control level.

In some embodiments, elevated or diminished levels of one or more factors or biomarkers in a biological sample may be used to determine the severity of the mast cell activity-associated disorder. In some embodiments, severity of the mast cell activity-associated disorder is the severity of MS. In some embodiments, elevated or diminished levels of one or more biomarkers are quantified in order to determine the severity and/or stage of the disorder.

### Methods for Identification of Agents Useful for Treatment of Mast Cell Activity-Associated Disorders

The present invention provides, among other things, methods for identifying agents that are useful in the treatment of mast cell activity-associated diseases, disorders and/or conditions. As such, the agents identified in accordance with the present invention may be useful in the treatment of mast cell activity-associated disorders, for example MS. The methods involve testing such agents against any of the mast cell activities disclosed herein and ascertaining the degree of protection provided by such agents against mast cell activity-associated diseases, disorders and/or conditions. In some embodiments, the invention contemplates assessing the candidate agent for modulation of: degranulation, transgranulation, level and/or location of a mast cell activity factor, level of a mast cell activity factor gene product, localization or migration of mast cells within a tissue or organ, degree or type of interaction with other cell types, degree or type of interaction with one or more particular proteins (e.g., myelin) or sites (e.g., basal lamina of the BBB) and combinations thereof.

stabilizing mast cell activity. For instance, the agent may stabilize the mast cell activity by returning the mast activity to a normal physiological state such that the disorder is treated. In some embodiments, a candidate agent may be assessed for the ability to inhibit mast cell proliferation, inhibit mast cell migration, inhibit release of cytokines, inhibit release of lipid derived metabolites, inhibit release of granule-associated metabolites, enhance hydration of a subject, reduce inflammation and combinations thereof. The agents may be tested for any of these activities in vitro or in vivo, for example in a mouse model of a mast cell activity-associated disorder.

In some embodiments, assessing the candidate agent for a presence, level and/or location of a mast cell activity occurs by comparing presence, level and/or location of mast cell activity for the candidate agent to a presence, level and/or location of mast cell activity for a control agent. The control agent may be, for example, phosphate-buffered saline, a placebo, water, vehicle, carrier. In some embodiments, level of mast cell activity of the candidate agent is more than 20%, 30%, 40%, 50%, 60%, 70%, 80%, 90%, 1-fold, 1.2-fold, 1.5-fold, 1.75-fold, 2-fold, 2.25-fold, 2.5-fold, 2.75-fold, 3-fold, 4-fold, 5-fold, 10-fold or 100-fold higher or lower than the level of mast cell activity for the control agent. Thus, the claimed method is useful for identifying agents that can treat mast cell associated diseases, disorders and/or conditions.

### Kits

In some embodiments, the present disclosure provides kits for determining and detecting biomarkers as described herein. In some embodiments, a kit is useful for determining a mast cell activity-associated disorder biomarker in a subject. In some embodiments, a kit comprises agents for determining a presence, level and/or location of one or more particular mast cell activity factors. In some embodiments, one or more particular mast cell activity factors have been determined to contribute to a mast cell activity biomarker for a mast cell activity-associated disorder, for example, MS susceptibility.

The present invention provides kits comprising various reagents and materials useful for carrying out inventive methods according to the present invention. The diagnosis and treatment procedures described herein may be performed by diagnostic laboratories, experimental laboratories, or practitioners. The invention provides kits which can be used in these different settings.

For example, materials and reagents for determining factors and biomarkers, measuring factor and biomarker levels (e.g., protein, lipids, histamine or nucleic acid levels), diagnosing mast cell activity-associated disorders, identifying subtypes, characterizing severity, staging the disorders, and/or monitoring treatment response in a subject according to the inventive methods may be assembled together in a kit. In certain embodiments, an inventive kit comprises at least one or more reagents that specifically detects protein, lipids, histamine or nucleic acid expression levels of one or more factors or biomarkers (e.g., those selected from Table 1), and instructions for using the kit according to a method of the invention.

Each kit may preferably comprise the reagent which renders the procedure specific. Thus, for detecting/quantifying a protein marker (or an analog or fragment thereof), the reagent that specifically detects expression levels of the protein may be an antibody that specifically binds to the protein marker (or analog or fragment thereof). For detecting/quantifying a nucleic acid molecule comprising a polynucleotide sequence coding a biomarker, the reagent that specifically detects expression levels may be a nucleic acid probe complementary to the polynucleotide sequence (e.g., cDNA or an oligonucleotide). The nucleic acid probe may or may not be immobilized on a substrate surface (e.g., beads, a microarray, and the like).

Kits or other articles of manufacture according to the invention may include one or more containers to hold various reagents. Suitable containers include, for example, bottles, vials, syringes (e.g., pre-filled syringes), ampules. The container may be formed from a variety of materials such as glass or plastic.

In some embodiments, kits of the present invention may include suitable reference levels or reference samples for determining reference levels as described herein. In some embodiments, kits of the invention may include instructions for using the kit according to one or more methods of the invention and may comprise instructions for processing the biological sample obtained from the subject and/or for performing the test, instructions for interpreting the results as well as a notice in the form prescribed by a governmental agency (e.g., FDA) regulating the manufacture, use or sale of pharmaceuticals or biological products.

The invention will be more fully understood by reference to the following examples. They should not, however, be construed as limiting the scope of the invention. All literature citations are incorporated by reference.

### EXEMPLIFICATION

### Example 1: Dural Mast Cell Interaction with T Cells in EAE

This example demonstrates exploration of dural mast cell interaction with T cells in the experimental autoimmune encephalomyelitis (EAE) animal model. The transcriptomic signature of T cells derived from draining deep cervical lymph nodes in mice with and without mast cells is compared to the transcriptomic signature of T cells from mice subject to induced mast cell degranulation. This example also demonstrates a comparison of B cell and monocyte profiles between the various groups of mice.

EAE is induced in wild type (using the Mog35-55 EAE model), and mast cell deficient mice (sash/sash). Two additional experimental groups are (1) mast cell deficient mice (sash/sash) reconstituted with mast cells ensuring that the effects observed are due to mast cells and not due to side effects of the mutation leading to mast cell dysfunction; and (2) wild type mice treated locally (CSF) with a compound that causes degranulation of mast cells.

T cells, B cells and monocytes are isolated from 5 tissue compartments of the four experimental groups. The compartments include: meningeal spaces, meningeal lymphatic vessels, parenchyma, deep cervical lymph nodes and blood. Cells are isolated at 4 time points: i) 7 days post immunization (prior to clinical signs), ii) 10-12 days post immunization (appearance of first clinical signs), iii) 18-20 days post immunization (a peak of clinical signs), and iv) after 30 days post immunization, (clinical score is stabilized at its lowest mark).

Cells are isolated from at least 18 mice from each of the 4 treatment groups. Cells and tissues from 6 mice are pooled into one sample, i.e., 3 samples for each condition for each organ, and analyzed by deep RNA sequencing. The data from the RNA sequencing studies is analyzed to identify functional differences in T cells exposed to normal or degranulated mast cells as compared to cells not exposed to mast cells (i.e., T cells from mast cell deficient mice). Unique biomarkers on the T cells are identified.

In parallel, deep RNA sequencing is performed on RNA extracted from T cells isolated from blood samples obtained from of MS patients. The results from analysis of human and mouse T cells are compared to assess expression of similar markers, or set of markers predictive of pathogenicity.

### Example 2: Comparison of Transcriptomic Signatures from Human MS Peripheral T Cells and T Cells from Mice with Induced Mast Cell Degranulation

This example demonstrates comparison of RNA sequencing derived transcriptomic signatures in human MS peripheral T cells to those obtained from the mouse studies in Example 1.

Blood samples from two groups of MS patients enrolled in the CLIMB study (Comprehensive Longitudinal Investigations in MS) are analyzed. The groups include patients with 1) early MS: samples collected from MS patients within one year of disease onset (n=10) and 2) relapse: samples collected from MS patients within 7 days prior to relapse (n=10). The control group for the first experimental group (early MS) is age and sex-matched healthy controls (n=10). The control group for the second experimental group (relapse MS) is the same patient at remission.

T cells are isolated from peripheral blood mononuclear cells (PBMC) using MACsBead separation. T cells are sorted from the PBMCs and then further sorted to isolate the naïve, effector, central and effector memory cell populations using CD45 and CCR7 antibodies and flow cytometry. T cell transcriptional profiles are derived using RNA-seq and variant calling. The transcriptional profiles are compared to the profiles obtained from the mouse studies in Example 1.

B cells and monocytes are also isolated from PBMC. B cell and monocyte transcriptional profiles are derived using RNA-seq and variant calling. The transcriptional profiles are compared to the profiles obtained from the mouse studies in Example 1.

### Example 3: Evaluation of In Vitro Triggers of Mast Cell Activity and Induction of T Cell Encephalitogenicity by Hormones

This example demonstrates that hormones enhance the triggering of mast cell degranulation, and secondarily enhance the encephalitogenic phenotype of T cells. In particular, the effect of sex hormones and stress hormones on mast cell degranulation and induction of T cell encephalitogenicity is demonstrated through analysis of the RNA-Seq transcriptomic profiles.

Cortisol-stimulated mast cells are incubated with T cell. The T cells are collected and RNA extracted. The RNA is analyzed by RNA-Seq and the profile is compared to naïve T cells. Estradiol-stimulated mast cells are incubated with T cell. The T cells are collected and RNA extracted. The RNA is analyzed by RNA-Seq and the profile is compared to naïve T cells. Progesterone-stimulated mast cells are incubated with T cell. The T cells are collected and RNA extracted. The RNA is analyzed by RNA-Seq and the profile is compared to naïve T cells.

### Example 4: Mast Cell Activity and Degranulation Factors are Increased at the Time of MS Relapse

This example demonstrates that mast cells activation and degranulation factors are increased at the time of an MS relapse and increased relative to periods of remission.

Adult patients with RRMS are enrolled in the study to determine if there is an increase in mast cell activity and degranulation factors at the time of an acute MS relapse. Inclusion criteria include: 1) RRMS meeting McDonald criteria, 2) age 18-40 years, 3) enrollment in the CLIMB study, 4) within 7 days of relapse-onset, as confirmed by study physician, 5) no steroid treatment within 30 days prior to relapse, 6) acceptable platform DMTs: INF, GA, Tysabri, Gilenya. A total of 30 subjects are studied.

Remission period samples are collected 3-6 months after relapse sample. The sample is not collected within 30 days of prior steroid treatment. The patient is receiving the same platform DMT as during the period of relapse.

Within 7 days of an acute relapse, patients present for an intravenous infusion of steroids, and blood and urine samples are collected. Three to six months later, a second blood and 24-hour urine sample is collected. Blood and urine samples from healthy control individuals are also collected. CSF samples are collected.

The serum is isolated from the blood and tested for cytokines (IL-4, IL-5, IL-6, IL-8, IL-17, IL-33, TNF-α, eosinophilic chemotactic factor), tryptase, chymase, histamine, heparin, chromogranin A, leukotriene E₄, prostaglandins (prostaglandin E₂, prostaglandin D₂, 11β-PGF_{2α}, tetranor-PGDM and other prostaglandin metabolites), CRH, cortisol and IgE. A metabolomics profile is performed on the serum sample to determine the complete set of small-molecule metabolites (such as metabolic intermediates, hormones and other signaling molecules, and secondary metabolites) found within the sample.

RNA is isolated from the whole blood sample and RNA sequencing is performed. The sample is evaluated for mast cell activation marker transcripts include CD117 which is also known as mast/stem cell growth factor receptor or c-Kit. CSF samples are analyzed for PGE-2, leukotrienes, CSF cellular transcripts and by metabolomic profiling. Levels of Prostaglandin E₂, leukotrienes, 11-β-prostaglandin F2-α and N-methylhistamine are measured in the urine samples. A metabolomics profile is performed on the urine sample to determine the complete set of small-molecule metabolites (such as metabolic intermediates, hormones and other signaling molecules, and secondary metabolites) found within the sample.

Levels of the biomarkers present in the blood, urine and CSF samples collected from the healthy controls, the MS patients at relapse and the MS patients at remission are compared.

### Example 5: Evaluation of Mast Cell Activity and Degranulation Triggers in Association with MS Relapse Severity

The Example will demonstrate that the degree of mast cell degranulation is associated with the severity of MS relapse.

The severity of the MS relapse is evaluated by the patient's physician. The relapse recovery is evaluated by the patient's physician. The levels of mast cell activity factors in blood and urine determined in Example 4 are correlated with the clinical findings during relapse and recovery.

### Example 6: Validation of Mast Cell-Induced T Cell Activity Transcriptomes

This example demonstrates the association of transcriptomes identified in the prior Examples with gene expression in the BBB and lymphatic vasculature. CRISPR-Cas9 systems, as well as animal knockout models are utilized.

### Example 7: Genetic Markers Related to RNA-Seq Transcriptomic Signatures in MS

This example demonstrates genetic markers related to the RNA-seq transcriptome signatures in MS. The presence of mast-cell associated allelic variants (e.g., CYP21A2) in MS patients versus healthy controls is demonstrated using whole genome sequencing.

### Example 8: Mast Cell Activity Factors and Biomarkers in Cancer

This example demonstrates identification of mast cell activity factors and biomarkers in cancer.

Tumor samples and matched normal tissue samples from cancer patients are obtained. Samples from the following cancer types are studied: breast bladder cancer, breast cancer, carcinoid, colon cancer, rectal cancer, glioblastoma, liver cancer, lung cancer, non-small cell lung cancer, chronic lymphocytic leukemia, Hodgkins' lymphoma, non-Hodgkin's lymphoma, malignant melanoma, multiple myeloma, neuroblastoma, ovarian cancer, pancreatic cancer, prostate cancer, renal cell carcinoma, throat cancer and uterine cancer. RNA, DNA and mast cell activity factors identified in Table 1 are extracted from the tumor sample and normal tissue sample. RNA is analyzed by RNA-Seq and the transcriptome profile of the tumor sample and matched normal tissue sample are compared and relevant biomarkers are identified. DNA is analyzed by whole exome sequencing and for specific variants including allelic variants of genes of the P450 enzyme family (e.g., CYP21A2). Results obtained from the tumor sample and match normal tissue sample are compared and relevant biomarkers are identified. The presence, level and/or location of mast cell activity factors are determined in the tumor sample and matched normal tissue sample. The results obtained from the tumor sample and match normal tissue sample are compared and relevant biomarkers are identified.

### Example 9: Experimental Autoimmune Encephalomyelitis (EAE) Study in SJL Mice

This example demonstrates detection of mast cell activity factors and biomarkers in a mouse model of multiple sclerosis.

The presence of PGDM, a metabolite of PGD₂, and histamine and/or N-methylhistamine in urine and serum of mice that develop EAE by active immunization was measured. EAE was induced by myelin proteolipid protein (PLP)₁₃₉₋₁₅₁/Complete Freund's Adjuvant (CFA) immunization of SJL mice.

TABLE 2
Study Design
MODEL

| | |
|---|---|
| Description | EAE in SJL mice |
| Animal strain(s) & gender(s) | SJL mice, females |
| Day 0 | Day of immunization |
| Study length | 16 days (from Day 0 until Day 15) |

ANIMALS & GROUPS

| | |
|---|---|
| Total number of animals | 23 |
| Source of animals | The Jackson Laboratory (breeder) |
| Age at start of study (Day 0) | 8 to 9 weeks |
| Number of groups | 1 |
| Group size | 23 animals (per Table 3) |
| Group assignment day(s) | Day 0 |

READOUTS

| | |
|---|---|
| Scoring starts | Day 9 after immunization |
| Scoring ends | Day 15 |
| Scoring frequency | Daily |
| Weighing starts | Day 0 |
| Weighing ends | End of study |
| Weighing frequency | 3x/week (Monday, Wednesday, Friday) |
| Tissue collection & analysis | Collect urine, serum, brain, spinal cord, |
| | cervical LN every day from 3 mice |
| | (per Table 3) |

### EAE Induction

Mice were acclimated at the testing facility for 4 weeks prior to the start of the study. EAE was induced in all mice by immunization with PLP₁₃₉₋₁₅₁/CFA. Specifically, mice were injected subcutaneously at four sites in the back with emulsion containing PLP₁₃₉₋₁₅₁ from Hook Kit PLP₁₃₉₋₁₅₁/CFA Emulsion, catalog number EK-0120 (Hooke Laboratories, Lawrence Mass.). Two sites of injection were in the upper back approximately 1 cm caudal of the neck line. Two more sites were in the lower back approximately 2 cm cranial of the base of the tail. The injection volume was 0.05 mL at each site.

All 23 mice were in a single group. Mice were scored for signs of paralysis (EAE) daily starting on Day 9 after immunization. Mice were sacrificed according to the schedule below in Table 3.

TABLE 3
Sacrifice schedule

| Sacrifice | Number of animals |
|---|---|
| Day 4 | 3 |
| Day 5 | 3 |
| Day 6 | 3 |
| Day 7 | 3 |
| Day 8 | 3 |
| Day 9 | 3 |
| Day 13 | 5 |

On each of Days 4, 5, 6, 7, 8, and 9, three (3) mice were terminated and tissue collected as described below in Table 4. EAE onset was expected on Day 11, therefore the mice were expected to be without signs of EAE through day 9. The 5 remaining mice were terminated on their first or second day of EAE appearance (day 13).

### Tissue Collection

The following tissues and fluids were collected from each mouse at the time of sacrifice and as provided in Table 4. Four naïve mice (i.e., no EAE induction) were also collected for urine and serum to provide a baseline control.

Urine

Serum

Cervical lymph nodes (2) frozen in OCT

Brain, frozen in OCT

Spinal cord fixed in 4% formalin for 24 hours, then frozen in OCT

TABLE 4
Specimen collection

| | | | Day 3 after | Day after | | | | | |
|---|---|---|---|---|---|---|---|---|---|
| | | | immunization | Immunization | | | | Cervical | |
| Cage | Mouse | Mouse | urine | collected/ | | Blood | Brain | lymph | Spinal |
| # | # | ID# | collected | terminated | Urine | (serum) | (whole) | nodes | cord |
| 1 | 1 | 1-1 | | 4 | + | + | + | + | + |
| | 2 | 1-2 | | 4 | | + | + | + | + |
| | 3 | 1-3 | | 4 | | + | + | + | + |
| | 4 | 1-4 | | 5 | + | + | + | + | + |
| | 5 | 1-5 | | 6 | + | + | + | + | + |
| | 6 | 1-6 | | 13 | + | + | | | |
| 2 | 1 | 2-1 | | 8 | + | + | + | + | + |
| | 2 | 2-2 | | 13 | + | + | | | |
| | 3 | 2-3 | + | 8 | + | + | + | + | + |
| | 4 | 2-4 | | 13 | + | + | | | |
| | 5 | 2-5 | + | 13 | + | + | | | |
| | 6 | 2-6 | + | | | | | | |
| 3 | 1 | 3-1 | + | 5 | + | + | + | + | + |
| | 2 | 3-2 | + | 5 | + | + | + | + | + |
| | 3 | 3-3 | + | 7 | + | + | + | + | + |
| | 4 | 3-4 | | 9 | + | + | + | + | + |
| | 5 | 3-5 | + | 8 | + | + | + | + | + |
| | 6 | 3-6 | | 9 | + | + | + | + | + |
| 4 | 1 | 4-1 | + | 6 | + | + | + | + | + |
| | 2 | 4-2 | + | 7 | + | + | + | + | + |
| | 3 | 4-3 | + | 7 | + | + | + | + | + |
| | 4 | 4-4 | + | 6 | + | + | + | + | + |
| | 5 | 4-5 | | 9 | + | + | + | + | + |

### Analysis of Fluids (Urine and Serum)

A colorimetric ELISA for N-methylhistamine and/or histamine (Enzo, ENZ-KIT140) was performed on each urine and serum sample. The ELISA method was sensitive down to 0.03 ng/mL. Each sample was tested in unicate due to small total sample size.

A competitive ELISA for tetranor-PGDM (a prostaglandin D₂ metabolite) (Cayman, Cat#501001) was performed on each urine and serum sample. Tetranor-PGDM is a major metabolite of PGD₂ and is found in human and mouse urine. The ELISA method converts tetranor-PGDM to a stable derivative, tetranor-PGJM, that can be quantified. The method detects from 6.4 to 4,000 pg tetranor-PGDM/mL.

### Readouts

Mice were scored daily for EAE from Day 9 until the end of the study, and body weight was measured three times/week (Monday, Wednesday, and Friday), starting on Day 0. The last day of the study was Day 15. Scoring was performed blind, by a person unaware of both treatment and of previous scores for each mouse. Readout was EAE scores on the scale 0-5 in 0.5 unit increments, and changes in body weight (Table 5).

TABLE 5
EAE scoring

| Score | Clinical Observations |
|---|---|
| 0.0 | No obvious changes in motor function compared to non-immunized mice. |
| | When picked up by base of tail, the tail has tension and is erect. Hind legs are usually |
| | spread apart. When the mouse is walking, there is no gait or head tilting. |
| 0.5 | Tip of tail is limp. |
| | When picked up by base of tail, the tail has tension except for the tip. Muscle |
| | straining is felt in the tail, while the tail continues to move. |
| 1.0 | Limp tail. |
| | When picked up by base of tail, instead of being erect, the whole tail drapes over the |
| | finer. Hind legs are usually spread apart. No signs of tail movement are observed. |
| 1.5 | Limp tail and hind leg inhibition. |
| | When picked up by base of tail, the whole tail drapes over the finger. When the |
| | mouse is dropped on a wire rack, at least one hind leg falls through consistently. |
| | Walking is very slightly wobbly. |
| 2.0 | Limp tail and weakness of hind legs. |
| | When picked up by base of tail, the legs are not spread apart, but held closer together. |
| | When the mouse is observed walking, it has a clearly apparent wobbly walk. One foot |
| | may have toes dragging, but the other leg has not apparent inhibitions of movement. |
| | OR |
| | Mouse appears to be a score 0.0, but there obvious signs of head tilting when the walk |
| | is observed. The balance is poor. |
| 2.5 | Limp tail and dragging of hind legs. |
| | Both hind legs have some movement, but both are dragging at the feet (mouse trips on |
| | hind feet). |
| | OR |
| | No movement in one leg/completely dragging one leg, but movement in the other leg. |
| | OR |
| | EAE severity appears mild when picked up (as score 0.0-1.5), but there is a strong |
| | head tilt that causes the mouse to occasionally fall over. |
| 3.0 | Limp tail and complete paralysis of hind legs (most common). |
| | OR |
| | Limp tail and almost complete paralysis of hind legs. One or both hind legs are able to |
| | paddle, but neither hind leg is able to move forward of the hind hip. |
| | OR |
| | Limp tail with paralysis of one front and one hind leg. |
| | OR |
| | ALL of: |
| | Severe head tilting, |
| | Walking only along the edges of the cage, |
| | Pushing against the cage wall, |
| | Spinning when picked up by base of tail. |
| 3.5 | Limp tail and complete paralysis of hind legs. In addition to: |
| | Mouse is moving around the cage, but when placed on its side, is unable to right itself. |
| | Hind legs are together on one side of body. |
| | OR |
| | Mouse is moving around the cage, but the hind quarters are flat like a pancake, giving |
| | the appearance of a hump in the front quarters of the mouse. |
| 4.0 | Limp tail, complete hind leg and partial front leg paralysis. |
| | Mouse is minimally moving around the cage but appears alert and feeding. Often |
| | euthanasia is recommended after the mouse scores 4.0 for 2 days. However, with daily |
| | s.c. fluids most C57BL/6 mice may recover to 3.5 or 3.0, while SJL mice may fully |
| | recover even if they reach score 4.0 at the peak of disease. When the mouse is |
| | euthanized because of severe paralysis, a score of 5.0 is entered for that mouse for the |
| | rest of the experiment. |
| 4.5 | Complete hind and partial front leg paralysis, no movement around the cage. Mouse is |
| | not alert. |
| | Mouse has minimal movement in the front legs. The mouse barely responds to contact. |
| | Euthanasia is recommended. When the mouse is euthanized because of severe |
| | paralysis, a score of 5.0 is entered for that mouse for the rest of the experiment. |
| 5.0 | Mouse is spontaneously rolling in the cage (euthanasia is recommended). |
| | OR |
| | Mouse is found dead due to paralysis. |
| | OR |
| | Mouse is euthanized due to severe paralysis. |

### Results

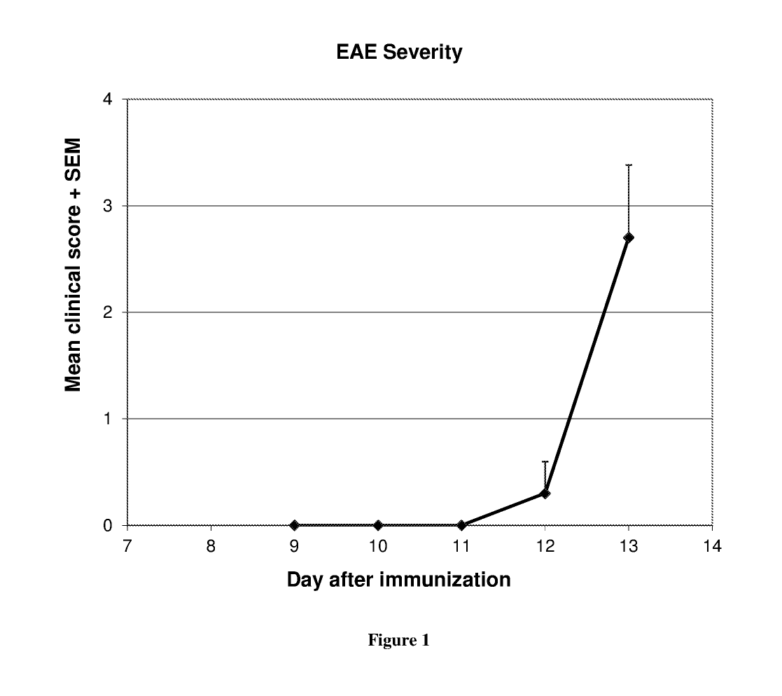

EAE scores were measured in the 5 mice sacrificed on day 13 of the study (Table 6). Four of the 5 mice developed significant signs of EAE by 13 days after immunization (FIG. 1).

TABLE 6
EAE scores

| | | | | | | | | | Mean | | Score | |
|---|---|---|---|---|---|---|---|---|---|---|---|---|
| Cage | Mouse | Mouse | Day | | | | | | day of | Incidence | at | End |
| # | # | ID# | 9 | 10 | 11 | 12 | 13 | MMS | onset | of EAE | onset | score |
| 1 | 1 | 1-1 | | | | | | | | | | |
| | 2 | 1-2 | | | | | | | | | | |
| | 3 | 1-3 | | | | | | | | | | |
| | 4 | 1-4 | | | | | | | | | | |
| | 5 | 1-5 | | | | | | | | | | |
| | 6 | 1-6 | 0.0 | 0.0 | 0.0 | 0.0 | 3.5 | 3.5 | 13 | | 3.5 | 3.5 |
| 2 | 1 | 2-1 | | | | | | | | | | |
| | 2 | 2-2 | 0.0 | 0.0 | 0.0 | 0.0 | 3.0 | 3.0 | 13 | | 3.0 | 3.0 |
| | 3 | 2-3 | | | | | | | | | | |
| | 4 | 2-4 | 0.0 | 0.0 | 0.0 | 0.0 | 3.5 | 3.5 | 13 | | 3.5 | 3.5 |
| | 5 | 2-5 | 0.0 | 0.0 | 0.0 | 1.5 | 3.5 | 3.5 | 12 | | 1.5 | 3.5 |
| | 6 | 2-6 | 0.0 | 0.0 | 0.0 | 0.0 | 0.0 | 0.0 | | | | 0.0 |
| 3 | 1 | 3-1 | | | | | | | | | | |
| | 2 | 3-2 | | | | | | | | | | |
| | 3 | 3-3 | | | | | | | | | | |
| | 4 | 3-4 | | | | | | | | | | |
| | 5 | 3-5 | | | | | | | | | | |
| | 6 | 3-6 | | | | | | | | | | |
| 4 | 1 | 4-1 | | | | | | | | | | |
| | 2 | 4-2 | | | | | | | | | | |
| | 3 | 4-3 | | | | | | | | | | |
| | 4 | 4-4 | | | | | | | | | | |
| | 5 | 4-5 | | | | | | | | | | |
| | | Average | 0.00 | 0.00 | 0.00 | 0.30 | 2.70 | 2.70 | 12.8 | 17.4% | 2.88 | 2.70 |
| | | Std Dev | 0.00 | 0.00 | 0.00 | 0.67 | 1.52 | 1.52 | 0.5 | | 0.95 | 1.52 |
| | | SEM | 0.00 | 0.00 | 0.00 | 0.30 | 0.68 | 0.68 | 0.3 | | | |

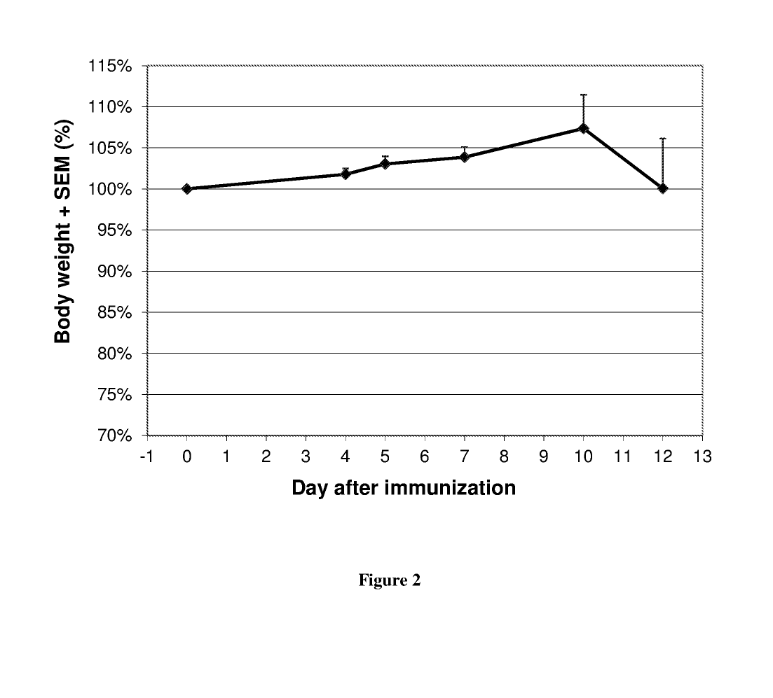

The body weights of the mice were measured on days 0, 4, 5, 7, 10 and 12 of the study (Table 7) and the percent body weight, relative to body weight on day 0 of the study, was determined (Table 8 and FIG. 2).

TABLE 7
Body weight

| Cage | Mouse | Mouse | Day | | | | | | | |
|---|---|---|---|---|---|---|---|---|---|---|
| # | # | ID# | 0 | 0 | 0 | 4 | 5 | 7 | 10 | 12 |
| 1 | 1 | 1-1 | 19.8 | 19.6 | 19.7 | 20.6 | | | | |
| | 2 | 1-2 | 19.9 | 19.8 | 19.9 | 20.6 | | | | |
| | 3 | 1-3 | 18.6 | 18.4 | 18.5 | 18.1 | | | | |
| | 4 | 1-4 | 19.3 | 19.3 | 19.3 | 20.5 | 21.1 | | | |
| | 5 | 1-5 | 18.4 | 18.2 | 18.3 | 18.5 | 18.8 | | | |
| | 6 | 1-6 | 21.0 | 21.2 | 21.1 | 21.2 | 21.6 | 22.0 | 21.6 | 19.4 |
| 2 | 1 | 2-1 | 18.6 | 18.5 | 18.6 | 19.8 | 20.2 | 20.2 | | |
| | 2 | 2-2 | 18.9 | 18.8 | 18.9 | 20.2 | 20.4 | 20.3 | 21.0 | 20.4 |
| | 3 | 2-3 | 18.9 | 18.7 | 18.8 | 19.5 | 19.6 | 19.9 | | |
| | 4 | 2-4 | 19.5 | 19.5 | 19.5 | 19.7 | 20.4 | 21.3 | 22.1 | 20.2 |
| | 5 | 2-5 | 23.1 | 23.0 | 23.1 | 23.6 | 23.5 | 23.2 | 21.6 | 18.7 |
| | 6 | 2-6 | 18.0 | 17.7 | 17.9 | 17.7 | 18.7 | 19.0 | 20.7 | 20.6 |
| 3 | 1 | 3-1 | 19.0 | 18.8 | 18.9 | 19.0 | 18.3 | | | |
| | 2 | 3-2 | 18.8 | 18.6 | 18.7 | 17.5 | 18.4 | | | |
| | 3 | 3-3 | 19.7 | 19.6 | 19.7 | 20.7 | 21.7 | 21.4 | | |
| | 4 | 3-4 | 22.3 | 22.3 | 22.3 | 21.8 | 21.7 | 21.8 | | |
| | 5 | 3-5 | 18.9 | 18.7 | 18.8 | 18.9 | 18.8 | 19.5 | | |
| | 6 | 3-6 | 19.8 | 19.7 | 19.8 | 19.5 | 19.8 | 19.8 | | |
| 4 | 1 | 4-1 | 17.8 | 17.7 | 17.8 | 18.1 | 18.4 | | | |
| | 2 | 4-2 | 21.8 | 21.7 | 21.8 | 22.7 | 21.9 | 21.4 | | |
| | 3 | 4-3 | 20.3 | 20.1 | 20.2 | 19.5 | 19.5 | 19.4 | | |
| | 4 | 4-4 | 20.9 | 20.8 | 20.9 | 21.6 | 21.7 | | | |
| | 5 | 4-5 | 17.5 | 17.4 | 17.5 | 18.1 | 18.4 | 18.5 | | |
| | | Average | | | 19.5 | 19.9 | 20.1 | 20.6 | 21.4 | 19.9 |
| | | Std Dev | | | 1.5 | 1.6 | 1.5 | 1.3 | 0.6 | 0.8 |
| | | SEM | | | 0.3 | 0.3 | 0.3 | 0.4 | 0.2 | 0.4 |

TABLE 8
Percent body weight

| | | | | | | | | | End % |
|---|---|---|---|---|---|---|---|---|---|
| Cage | Mouse | Mouse | Day | | | | | | body |
| # | # | ID# | 0 | 4 | 5 | 7 | 10 | 12 | weight |
| 1 | 1 | 1-1 | 100.0% | 104.6% | | | | | 104.6% |
| | 2 | 1-2 | 100.0% | 103.8% | | | | | 103.8% |
| | 3 | 1-3 | 100.0% | 97.8% | | | | | 97.8% |
| | 4 | 1-4 | 100.0% | 106.2% | 109.3% | | | | 109.3% |
| | 5 | 1-5 | 100.0% | 101.1% | 102.7% | | | | 102.7% |
| | 6 | 1-6 | 100.0% | 100.5% | 102.4% | 104.3% | 102.4% | 91.9% | 91.9% |
| 2 | 1 | 2-1 | 100.0% | 106.7% | 108.9% | 108.9% | | | 108.9% |
| | 2 | 2-2 | 100.0% | 107.2% | 108.2% | 107.7% | 111.4% | 108.2% | 108.2% |
| | 3 | 2-3 | 100.0% | 103.7% | 104.3% | 105.9% | | | 105.9% |
| | 4 | 2-4 | 100.0% | 101.0% | 104.6% | 109.2% | 113.3% | 103.6% | 103.6% |
| | 5 | 2-5 | 100.0% | 102.4% | 102.0% | 100.7% | 93.7% | 81.1% | 81.1% |
| | 6 | 2-6 | 100.0% | 99.2% | 104.8% | 106.4% | 116.0% | 115.4% | 115.4% |
| 3 | 1 | 3-1 | 100.0% | 100.5% | 96.8% | | | | 96.8% |
| | 2 | 3-2 | 100.0% | 93.6% | 98.4% | | | | 98.4% |
| | 3 | 3-3 | 100.0% | 105.3% | 110.4% | 108.9% | | | 108.9% |
| | 4 | 3-4 | 100.0% | 97.8% | 97.3% | 97.8% | | | 97.8% |
| | 5 | 3-5 | 100.0% | 100.5% | 100.0% | 103.7% | | | 103.7% |
| | 6 | 3-6 | 100.0% | 98.7% | 100.3% | 100.3% | | | 100.3% |
| 4 | 1 | 4-1 | 100.0% | 102.0% | 103.7% | | | | 103.7% |
| | 2 | 4-2 | 100.0% | 104.4% | 100.7% | 98.4% | | | 98.4% |
| | 3 | 4-3 | 100.0% | 96.5% | 96.5% | 96.0% | | | 96.0% |
| | 4 | 4-4 | 100.0% | 103.6% | 104.1% | | | | 104.1% |
| | 5 | 4-5 | 100.0% | 103.7% | 105.4% | 106.0% | | | 106.0% |
| | | Average | 100.0% | 101.8% | 103.0% | 103.9% | 107.4% | 100.1% | 102.1% |
| | | Std Dev | 0.0% | 3.5% | 4.2% | 4.5% | 9.2% | 13.6% | 7.0% |
| | | SEM | 0.0% | 0.7% | 0.9% | 1.2% | 4.1% | 6.1% | 1.5% |

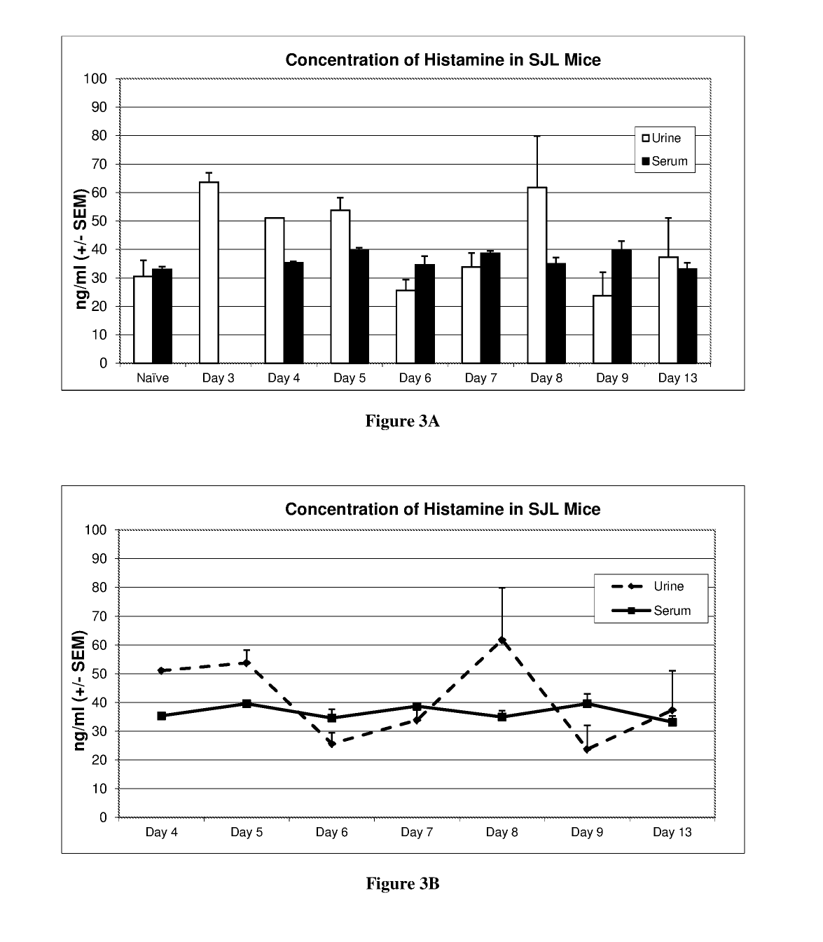

Urine was collected from the naïve mice and the EAE mice on day 3, 4, 5, 6, 7, 8, 9 and 13 post-immunization. Serum was collected from the naïve mice and the EAE mice on day 4, 5, 6, 7, 8, 9 and 13 post-immunization. Histamine was measured in urine and serum using an ELISA method as described above (FIGS. 3A and 3B). Data for individual mice is provided in Table 9.

TABLE 9
Histamine values

Histamine ng/mL
sample

| Group | Mouse ID | Urine | Serum |
|---|---|---|---|
| Naïve | N1 | 37.2 | 34.3 |
| | N2 | 13.9 | 35.0 |
| | N3 | 37.2 | 30.6 |
| | N4 | 34.0 | 32.0 |
| Average | | 30.6 | 33.0 |
| Std Dev | | 11.2 | 2.0 |
| SEM | | 5.6 | 1.0 |
| Day 3 | 2-3 | 86.9 | not collected |
| | 2-5 | 49.9 | not collected |
| | 2-6 | 68.4 | not collected |
| | 3-1 | 73.2 | not collected |
| | 3-2 | 58.4 | not collected |
| | 3-3 | 59.7 | not collected |
| | 3-5 | 65.4 | not collected |
| | 4-1 | 57.1 | not collected |
| | 4-2 | 54.5 | not collected |
| | 4-3 | 73.2 | not collected |
| | 4-4 | 53.3 | not collected |
| Average | | 63.6 | |
| Std Dev | | 11.0 | |
| SEM | | 3.3 | |
| t-test* | | 0.0002 | |
| Day 4 | 1-1 | 51.1 | 35.8 |
| | 1-2 | not collected | 34.3 |
| | 1-3 | not collected | 35.8 |
| Average | | 51.1 | 35.3 |
| Std Dev | | | 0.9 |
| SEM | | | 0.5 |
| t-test* | | | 0.1288 |
| Day 5 | 1-4 | 62.5 | 37.5 |
| | 3-1 | 51.1 | 40.1 |
| | 3-2 | 47.7 | 41.1 |
| Average | | 53.7 | 39.6 |
| Std Dev | | 7.7 | 1.8 |
| SEM | | 4.5 | 1.1 |
| t-test* | | 0.0287 | 0.0071 |
| Day 6 | 1-5 | 33.2 | 38.4 |
| | 4-1 | 21.1 | 28.6 |
| | 4-4 | 22.6 | 36.7 |
| Average | | 25.6 | 34.6 |
| Std Dev | | 6.6 | 5.2 |
| SEM | | 3.8 | 3.0 |
| t-test* | | 0.5337 | 0.5951 |
| Day 7 | 3-3 | 36.4 | 40.1 |
| | 4-2 | 40.7 | 37.5 |
| | 4-3 | 24.4 | 38.4 |
| Average | | 33.8 | 38.7 |
| Std Dev | | 8.4 | 1.3 |
| SEM | | 4.9 | 0.8 |
| t-test* | | 0.6920 | 0.0088 |
| Day 8 | 2-1 | 38.0 | 37.5 |
| | 2-3 | 49.9 | 30.6 |
| | 3-5 | 97.3 | 36.7 |
| Average | | 61.7 | 34.9 |
| Std Dev | | 31.4 | 3.8 |
| SEM | | 18.1 | 2.2 |
| t-test* | | 0.1183 | 0.4120 |
| Day 9 | 3-4 | 19.0 | 46.0 |
| | 3-6 | 12.4 | 34.3 |
| | 4-5 | 39.8 | 38.4 |
| Average | | 23.7 | 39.6 |
| Std Dev | | 14.3 | 5.9 |
| SEM | | 8.3 | 3.4 |
| t-test* | | 0.5083 | 0.0878 |
| Day 13 | 1-6 | 77.8 | 30.6 |
| | 2-2 | 18.2 | 29.2 |
| | 2-4 | 29.9 | 33.5 |
| | 2-5 | 23.3 | 39.2 |
| Average | | 37.3 | 33.1 |
| Std Dev | | 27.4 | 4.4 |
| SEM | | 13.7 | 2.2 |
| t-test* | | 0.6651 | 0.9534 |

*t-test comparison of EAE group to naïve group (p < 0.05 was considered significant).

A significant increase in urine histamine/N-methylhistamine levels, as compared to levels in naïve mice, was detected at day 3 and day 5. A significant increase in serum histamine levels, as compared to levels in naïve mice, was detected at day 5 and day 7.

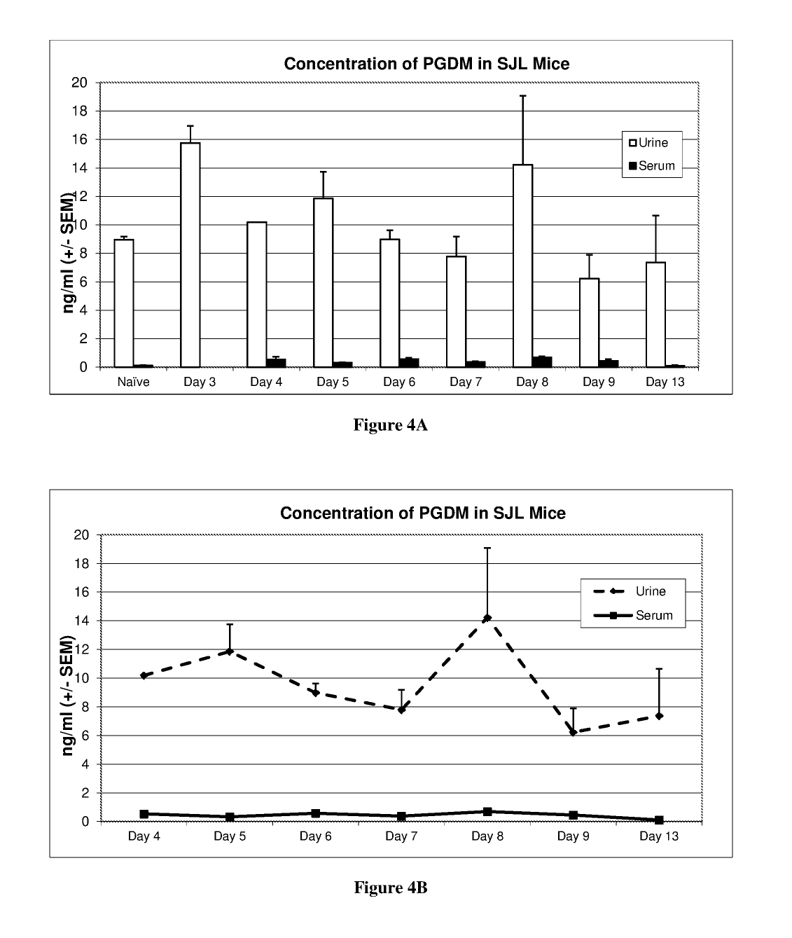

PGDM was measured in urine and serum using an ELISA method as described above (FIGS. 4A and 4B). Data for individual mice is provided in Table 10.

TABLE 10
PGDM values

PGDM ng/mL
sample

| Group | Mouse ID | Urine | Serum |
|---|---|---|---|
| Naïve | N1 | 8.622 | 0.148 |
| | N2 | 9.568 | 0.104 |
| | N3 | 8.622 | 0.050 |
| | N4 | 8.989 | 0.164 |
| Average | | 8.950 | 0.117 |
| Std Dev | | 0.447 | 0.051 |
| SEM | | 0.2 | 0.0 |
| Day 3 | 2-3 | 26.544 | not collected |
| | 2-5 | 10.399 | not collected |
| | 2-6 | 14.352 | not collected |
| | 3-1 | 16.261 | not collected |
| | 3-2 | 14.962 | not collected |
| | 3-3 | 13.483 | not collected |
| | 3-5 | 14.352 | not collected |
| | 4-1 | 15.926 | not collected |
| | 4-2 | 15.598 | not collected |
| | 4-3 | 15.926 | not collected |
| | 4-4 | 15.598 | not collected |
| Average | | 15.764 | |
| Std Dev | | 3.939 | |
| SEM | | 1.188 | |
| t-test* | | 0.0050 | |
| Day 4 | 1-1 | 10.185 | 0.271 |
| | 1-2 | not collected | 0.905 |
| | 1-3 | not collected | 0.410 |
| Average | | 10.185 | 0.529 |
| Std Dev | | | 0.333 |
| SEM | | | 0.192 |
| t-test* | | | 0.0534 |
| Day 5 | 1-4 | 15.598 | 0.294 |
| | 3-1 | 10.399 | 0.347 |
| | 3-2 | 9.568 | 0.320 |
| Average | | 11.855 | 0.320 |
| Std Dev | | 3.268 | 0.027 |
| SEM | | 1.887 | 0.015 |
| t-test* | | 0.1293 | 0.0016 |
| Day 6 | 1-5 | 10.185 | 0.378 |
| | 4-1 | 7.934 | 0.720 |
| | 4-4 | 8.804 | 0.609 |
| Average | | 8.974 | 0.569 |
| Std Dev | | 1.135 | 0.174 |
| SEM | | 0.655 | 0.101 |
| t-test* | | 0.9702 | 0.0039 |
| Day 7 | 3-3 | 8.989 | 0.410 |
| | 4-2 | 9.371 | 0.300 |
| | 4-3 | 4.987 | 0.394 |
| Average | | 7.782 | 0.368 |
| Std Dev | | 2.428 | 0.059 |
| SEM | | 1.402 | 0.034 |
| t-test* | | 0.3759 | 0.0018 |
| Day 8 | 2-1 | 9.371 | 0.810 |
| | 2-3 | 9.371 | 0.609 |
| | 3-5 | 23.920 | 0.635 |
| Average | | 14.221 | 0.685 |
| Std Dev | | 8.400 | 0.109 |
| SEM | | 4.850 | 0.063 |
| t-test* | | 0.2515 | 0.0002 |
| Day 9 | 3-4 | 4.784 | 0.224 |
| | 3-6 | 4.311 | 0.622 |
| | 4-5 | 9.568 | 0.475 |
| Average | | 6.221 | 0.440 |
| Std Dev | | 2.908 | 0.201 |
| SEM | | 1.679 | 0.116 |
| t-test* | | 0.1145 | 0.0245 |
| Day 13 | 1-6 | 17.245 | 0.055 |
| | 2-2 | 4.402 | 0.064 |
| | 2-4 | 3.575 | 0.092 |
| | 2-5 | 4.222 | 0.190 |
| Average | | 7.361 | 0.100 |
| Std Dev | | 6.599 | 0.062 |
| SEM | | 3.299 | 0.031 |
| t-test* | | 0.6478 | 0.6983 |

*t-test comparison of EAE group to naïve group (p < 0.05 was considered significant).

A significant increase in urine PGDM levels, as compared to levels in naïve mice, was detected at day 3. A significant increase in serum PGDM levels, as compared to levels in naïve mice, was detected at days 5-9.

These data demonstrate that histamine (and its metabolite N-methylhistamine), and PGD₂ (and its metabolite tetranor-PGDM) are mast cell activity biomarkers of mast cell activity-associated disorders, for example MS. By determining the presence, level and/or location of one or more mast cell activity factors, such as histamine and PGD₂, in samples (e.g., urine or serum) of subjects suffering from or susceptible to a mast cell activity-associated disorder, the occurrence, incidence, severity, or therapeutic response of the mast cell activity-associated disorder may be determined.

### Example 10: Experimental Autoimmune Encephalomyelitis (EAE) Study in C57BL/6 Mice

This example demonstrates detection of mast cell activity factors and biomarkers in a mouse model of multiple sclerosis.

The presence of PGDM, a metabolite of PGD₂, and histamine and/or N-methylhistamine in urine and serum of mice that develop EAE as a result of adoptive cell transfer, was measured. In this EAE model, immunization only occurs in donor mice. Fully encephalitogenic cells were transferred into recipient mice, where they induced EAE (effector phase of EAE).

TABLE 11
Study Design
MODEL

| | |
|---|---|
| Description | EAE in C57BL/6 mice |
| Animal strain(s) & gender(s) | C57BL/6 mice, females |
| Day 0 | Day of cell tranfer |
| Study length | 16 days (from Day 0 until Day 15) |

ANIMALS & GROUPS

| | |
|---|---|
| Total number of animals | 3 |
| Source of animals | The Jackson Laboratory (breeder) |
| Age at start of study (Day 0) | 8 to 9 weeks |
| Number of groups | 1 |
| Group size | 3 animals |
| Group assignment day(s) | Day 0 |

READOUTS

| | |
|---|---|
| Scoring starts | Day 6 after immunization |
| Scoring ends | Day 15 |
| Scoring frequency | Daily |
| Weighing starts | Day 0 |
| Weighing ends | End of study |
| Weighing frequency | 3x/week (Monday, Wednesday, Friday) |
| | Collect urine, serum, brain, spinal cord, |
| Tissue collection & analysis | cervical LN from 3 mice |

### EAE Induction

In the adoptive transfer model of EAE, female C57BL/6 donor mice were immunized and used as a source of encephalitogenic T cells. Once donor mice developed an immune response to myelin oligodendrocyte glycoprotein (MOG) ₃₅₋₅₅/CFA emulsion (usually 11 days after immunization), they were sacrificed and their spleens harvested. Spleen cells were cultured in the presence of MOG₃₅₋₅₅ to activate encephalitogenic T cells, which were then transferred to female C57BL/6 recipient mice to induce EAE. The day of cell transferred was considered Day 0 of the study. EAE onset is normally 6 to 9 days after the cell transfer.

Three mice were in a single group. Mice were scored for signs of paralysis (EAE) daily starting on Day 6 after immunization. Mice were sacrificed on day 15 following cell transfer.

### Tissue Collection

The following fluids were collected from each mouse at the time of sacrifice and as provided in Table 12. Mouse 1-4 did not receive a cell transfer and served as the naïve control.

Urine

Serum

TABLE 12
Specimen collection

| | | | Day after cell | | |
|---|---|---|---|---|---|
| | | Mouse | transfer | | Blood |
| Cage # | Mouse # | ID# | collected | Urine | (serum) |
| 1 | 1 | 1-1 | 1 | + | + |
| | 2 | 1-2 | 1 | + | + |
| | 3 | 1-3 | 1 | + | + |
| | 4 | 1-4 | 1 | + | + |

### Analysis of Fluids (Urine and Serum)

A colorimetric ELISA for N-methylhistamine and/or histamine (Enzo, ENZ-KIT140) was performed on each urine and serum sample. The ELISA method was sensitive down to 0.03 ng/mL. Each sample was tested in unicate due to small total sample size.

A competitive ELISA for tetranor-PGDM (a prostaglandin D₂ metabolite) (Cayman, Cat#501001) was performed on each urine and serum sample. Tetranor-PGDM is a major metabolite of PGD₂ and is found in human and mouse urine. The ELISA method converts tetranor-PGDM to a stable derivative, tetranor-PGJM, that can be quantified. The method detects from 6.4 to 4,000 pg tetranor-PGDM/mL.

### Results

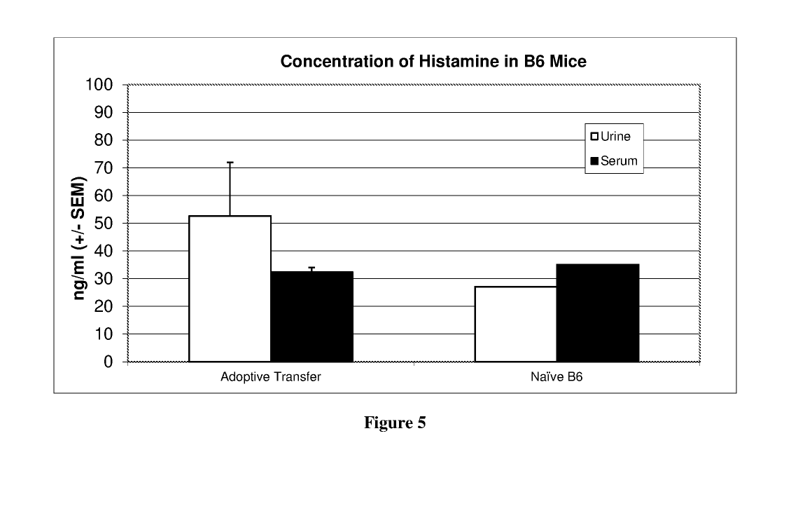

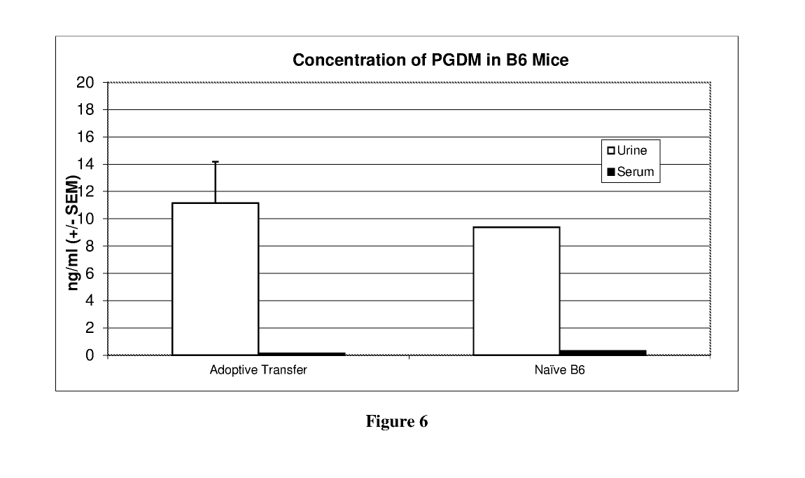

Urine and serum were collected from the naïve and EAE mice on day 15 post-cell transfer. Histamine/N-methylhistamine and PGDM were measured as described in Example 9 (FIGS. 5 and 6). Histamine data for individual mice is provided in Table 13.

TABLE 13
Histamine values

Histamine ng/mL
sample

| Group | Mouse ID | Urine | Serum |
|---|---|---|---|
| Adoptive Transfer | A1 | 91.2 | 32.7 |
| | A2 | 31.0 | 35.0 |
| | A3 | 35.6 | 29.2 |
| Average | | 52.6 | 32.3 |
| Std Dev | | 33.5 | 2.9 |
| SEM | | 19.3 | 1.7 |
| t-test* | | 0.2634 | 0.7467 |
| Naïve B6 | N1 B6 | 27.1 | 35.0 |

*t-test comparison of EAE group to naïve group (p < 0.05 was considered significant).

PGDM data for individual mice is provided in Table 14.

TABLE 14
PGDM values

PGDM ng/mL
sample

| Group | Mouse ID | Urine | Serum |
|---|---|---|---|
| Adoptive Transfer | A1 | 16.890 | 0.131 |
| | A2 | 9.975 | 0.133 |
| | A3 | 6.578 | 0.145 |
| Average | | 11.148 | 0.136 |
| Std Dev | | 5.255 | 0.008 |
| SEM | | 3.034 | 0.004 |
| t-test | | 0.4286 | 0.5452 |
| Naïve B6 | N1 B6 | 9.371 | 0.307 |

*t-test comparison of EAE group to naïve group (p < 0.05 was considered significant).

These data demonstrate that histamine (and its metabolite N-methylhistamine), and PGD₂ (and its metabolite tetranor-PGDM) are mast cell activity biomarkers of mast cell activity-associated disorders, for example MS. By determining the presence, level and/or location of one or more mast cell activity factors, such as histamine and PGD₂, in samples (e.g., urine or serum) of subjects suffering from or susceptible to a mast cell activity-associated disorder, the occurrence, incidence, severity, or therapeutic response of the mast cell activity-associated disorder may be determined.

### EQUIVALENTS

Those skilled in the art will recognize, or be able to ascertain using no more than routine experimentation, many equivalents to embodiments of the inventions described herein. The scope of the present invention is not intended to be limited to the above Description, but rather is as set forth in the following claims.

### SEQUENCE LISTING

| CYP21A2 cytochrome P450 family 21 subfamily A member 2 gene [Homo sapiens | |
|---|---|
| (human)] X58906.1 | |
| (SEQ ID NO: 1) | |
| 1 | tggggctctt gagctataag tggcacctca gggccctgac gggcgtcttg ccatgctgct |
| 61 | cctgggcctg ctgctgctgc tgcccctgct ggctggcgcc cgcctgctgt ggaactggtg |
| 121 | gaagctccgg agcctccacc tcccgcctct tgccccgggc ttcttgcact tgctgcagcc |
| 181 | cgacctccca atctatctgc ttggcctgac tcagaaattc gggcccatct acaggctcca |
| 241 | ccttgggctg caaggtgaga ggctgatctc gctctggccc tcaccatagg agggggcgga |
| 301 | ggtgacggag agggtcctct ctccgctgac gctgctttgg ctgtctccca gatgtggtgg |
| 361 | tgctgaactc caagaggacc attgaggaag ccatggtcaa aaagtgggca gactttgctg |
| 421 | gcagacctga gccacttacc tgtaagggct gggggcattt tttctttctt aaaaaaattt |
| 481 | ttttttaaga gatgggttct tgctatgctg cccaggctgg tcttaaattc ctagtctcaa |
| 541 | atgatcctcc cacctcagcc tcaagtgtga gccacctttg gggcatcccc aatccaggtc |
| 601 | cctggaagct cttggggggg catatctggt ggggagaaag caggggttgg ggaggccgaa |
| 661 | gaaggtcagg ccctcagctg ccttcatcag ttcccaccct ccagccccca cctcctcctg |
| 721 | cagacaagct ggtgtctagg aactacccgg acctgtcctt gggagactac tccctgctct |
| 781 | ggaaagccca caagaagctc acccgctcag ccctgctgct gggcatccgt gactccatgg |
| 841 | agccagtggt ggagcagctg acccaggagt tctgtgaggt aaggctgggc tcctgaggcc |
| 901 | acctcgggtc agcctcgcct ctcacagtag cccccgccct gcccgctgca cagcggcctg |
| 961 | ctgaactcac actgtttctc cacagcgcat gagagcccag cccggcaccc ctgtggccat |
| 1021 | tgaggaggaa ttctctctcc tcacctgcag catcatctgt tacctcacct tcggagacaa |
| 1081 | gatcaaggtg cctcacagcc cctcaggccc acccccagcc cctccctgag cctctccttg |
| 1141 | tcctgaactg aaagtactcc ctccttttct ggcaggacga caacttaatg cctgcctatt |
| 1201 | acaaatgtat ccaggaggtg ttaaaaacct ggagccactg gtccatccaa attgtggacg |
| 1261 | tgattccctt tctcagggtg aggacctgga gcctagacac ccctgggttg taggggagag |
| 1321 | gctggggtgg agggagaggc tccttcccac agctgcattc tcatgcttcc tgccgcagtt |
| 1381 | cttccccaat ccaggtctcc ggaggctgaa gcaggccata gagaagaggg atcacatcgt |
| 1441 | ggagatgcag ctgaggcagc acaaggtggg gactgtacgt ggacggcctc ccctcggccc |
| 1501 | acagccagtg atgctaccgg cctcagcatt gctatgaggc gggttctttt gcatacccca |
| 1561 | gttatgggcc tgttgccact ctgtactcct ctccccaggc cagccgctca gcccgctcct |
| 1621 | ttcaccctct gcaggagagc ctcgtggcag gccagtggag ggacatgatg gactacatgc |
| 1681 | tccaaggggt ggcgcagccg agcatggaag agggctctgg acagctcctg gaagggcacg |
| 1741 | tgcacatggc tgcagtggac ctcctgatcg gtggcactga gaccacagca aacaccctct |
| 1801 | cctgggccgt ggtttttttg cttcaccacc ctgaggtgcg tcctggggac aagcaaaagg |
| 1861 | ctccttccca gcaacctggc cagggcggtg ggcaccctca ctcagctctg agcactgtgc |
| 1921 | ggctggggct gtgcttgcct caccggcact caggctcact gggttgctga gggagcggct |
| 1981 | ggaggctggg cagctgtggg ctgctggggc aggactccac ccgatcattc cccagattca |
| 2041 | gcagcgactg caggaggagc tagaccacga actgggccct ggtgcctcca gctcccgggt |
| 2101 | cccctacaag gaccatgcac ggctgccctt gctcaatgcc accatcgccg aggtgctgcg |
| 2161 | cctgcggccc gttgtgccct tagccttgcc ccaccgcacc acacggccca gcaggtgact |
| 2221 | cccgagggtt ggggatgagt gaggaaagcc cgagcccagg gaggtcctgg ccagcctcta |
| 2281 | actccagccc ccttcagcat ctccggctac gacatccctg agggcacagt catcattccg |
| 2341 | aacctccaag gcgcccacct ggatgagacg gtctgggaga ggccacatga gttctggcct |
| 2401 | ggtatgtggg ggccgggggc ctgccgtcaa aatgtggtgg aggctggtcc ccgctgccgc |
| 2461 | tgaacgcctc cccacccacc tgtccacccg cccgcagatc gcttcctgga gccaggcaag |
| 2521 | aactccagag ctctggcctt cggctgcggt gcccgcgtgt gcctgggcga gccgctggcg |
| 2581 | cgcctggagc tcttcgtggt gctgacccga ctgctgcagg ccttcacgct gctgtcctcc |
| 2641 | ggggacgccc tgccctccct gcagcccctg ccccactgca gtgtcatcct caagatgcag |
| 2701 | cctttccaag tgcggctgca gccccggggg atgggggccc acagcccggg ccagagccag |
| 2761 | tgatggggca g |

```
CYP21A2 Protein Sequence P08686 (CP21A HUMAN)
(SEQ ID NO: 2)
                10 20 30 40 50
MLLLGLLLLP LLAGARLLWN WWKLRSLHLP PLAPGFLHLL QPDLPIYLLG
                60 70 80 90 100
LTQKFGPIYR LHLGLQDVVV LNSKRTIEEA MVKKWADFAG RPEPLTYKLV
              110 120 130 140 150
SKNYPDLSLG DYSLLWKAHK KLTRSALLLG IRDSMEPVVE QLTQEFCERM
              160 170 180 190 200
RAQPGTPVAI EEEFSLLTCS IICYLTFGDK IKDDNLMPAY YKCIQEVLKT
              210 220 230 240 250
WSHWSIQIVD VIPFLRFFPN PGLRRLKQAI EKRDHIVEMQ LRQHKESLVA
              260 270 280 290 300
GQWRDMMDYM LQGVAQPSME EGSGQLLEGH VHMAAVDLLI GGTETTANTL
              310 320 330 340 350
SWAVVFLLHH PEIQQRLQEE LDHELGPGAS SSRVPYKDRA RLPLLNATIA
              360 370 380 390 400
EVLRLRPVVP LALPHRTTRP SSISGYDIPE GTVIIPNLQG AHLDETVWER
              410 420 430 440 450
PHEFWPDRFL EPGKNSRALA FGCGARVCLG EPLARLELFV VLTRLLQAFT
              460 470 480 490
LLPSGDALPS LQPLPHCSVI LKMQPFQVRL QPRGMGAHSP GQNQ
```

We claim:

1. A method of treating a mast cell activity-associated disorder in a subject in need of treatment, the method comprising:
administering a mast cell activity inhibitor to a subject, wherein presence of a mast cell activity biomarker has been detected in a sample from the subject.

2. The method of claim 1, wherein the subject is diagnosed as suffering from, or susceptible to, the mast cell activity-associated disorder.

3. The method of claim 1, wherein the mast cell activity inhibitor is or comprises: a T-cell receptor ligand that competes with or inhibits one or more T-cell activating factors.

4. The method of claim 1, wherein the mast cell activity inhibitor is or comprises: a mast cell adhesion inhibitor.

5. The method of claim 1, wherein the mast cell activity inhibitor is or comprises: an inhibitor of mast cell degranulation products.

6. The method of claim 1, wherein the mast cell activity inhibitor is or comprises: a B-cell activity inhibitor.

7. The method of claim 1, wherein the mast cell activity inhibitor is or comprises: a gene therapy agent that corrects a mutation in the cytochrome P450 family.

8. The method of claim 1, wherein the sample is selected from the group consisting of whole blood, plasma, serum, urine, cerebrospinal fluid and lymphatic fluid.

9. A method of treating a mast cell activity-associated disorder in a subject in need of treatment, the method comprising:
administering one or both of:
a first agent selected from the group consisting of chondroitin, methylsulfonylmethane (MSM), glucosamine, an H₁ receptor antagonist, an H₂ receptor antagonist, and combinations thereof; and
a second agent selected from the group consisting of selective serotonin reuptake inhibitors (SSRIs), norepinephrine-dopamine reuptake inhibitors (NDRIs), and combinations thereof,
to the subject if presence of a mast cell activity biomarker has been detected in a sample from the subject, so that the subject is treated with the first and second agents in combination.

10. The method of claim 9, wherein the first agent and the second agent are administered at the same time or are administered sequentially.

11. A method of treating a mast cell activity-associated disorder in a subject in need of treatment, the method comprising:
administering to a subject a nanobot agent adapted to detect presence of a mast cell activity biomarker and to deliver a mast cell activity inhibitor, so that the mast cell activity inhibitor is administered when the biomarker is detected.

12. A method of diagnosing a subject as susceptible to MS relapse, the method comprising:
detecting a presence, level and/or location of a mast cell activity biomarker in the subject or in a sample from the subject;
predicting the occurrence of MS relapse based on the mast cell activity biomarker that is detected; and
administering a mast cell activity inhibitor to treat the MS relapse.

13. The method of claim 12, wherein the step of detecting comprises contacting the subject or sample with a molecular contrast agent to visualize mast cell degranulation.

14. The method of claim 12, wherein the sample is selected from the group consisting of whole blood, plasma, serum, urine, cerebrospinal fluid and lymphatic fluid.

15. A method of determining a mast cell activity biomarker of a mast cell activity-associated disorder, the method comprising steps of:
determining presence, level and/or location of one or more mast cell activity factors in samples of subjects suffering from or susceptible to the mast cell activity-associated disorder;
detecting a correlation between a determined presence, level and/or location of the one or more mast cell activity factors with incidence, severity, or therapeutic response of the mast cell activity-associated disorder, thereby establishing the determined presence, level and/or location of a mast cell activity biomarker for the incidence, severity, or therapeutic response.

16. The method of claim 15, wherein the correlated determined presence, level and/or location includes a plurality of data points, each representing presence, level and/or location of a different mast cell activity factor.

17. The method of claim 16, wherein at least one data point represents a level relative to an established threshold for a particular mast cell activity factor.

18. The method of claim 15, wherein the incidence, severity, or therapeutic response of the mast cell activity-associated disorder correlates with the presence, level and/or location of mast cell activity, mast cell proliferation, mast cell migration, immune cell localization, release of cytokines, release of lipid derived metabolites, release of granule-associated metabolites, hydration, inflammation and combinations thereof.

19. The method of claim 15, wherein the mast cell activity-associated disorder is MS.

20. The method of claim 19, wherein the incidence, severity, or therapeutic response of the MS correlates with the presence, level and/or location of mast cell activity, mast cell proliferation, mast cell migration, immune cell localization, release of cytokines, release of lipid derived metabolites, release of granule-associated metabolites, hydration, inflammation and combinations thereof.

21. The method of claim 15, wherein the subject is a mouse model of mast cell activity-associated disorder.

22. The method of claim 15, wherein the step of determining comprises visualizing T-cell activation factors, B-cell activation factors and/or mast cell degranulation factors in tissue of the mouse model.

23. The method of claim 15, wherein the sample is selected from the group consisting of whole blood, plasma, serum, urine, cerebrospinal fluid and lymphatic fluid.

24. A method of detecting a mast cell activity biomarker of a mast cell activity-associated disorder,
obtaining a sample from a human patient:
detecting in the sample presence, level and/or location of one or more mast cell activity factors determined to be a mast cell activity biomarker of the mast cell activity-associated disorder.

25. The method of claim 24, wherein the method further comprises a step of comparing the presence, level and/or location of one or more mast cell activity factors with a reference presence, level and/or location of one or more mast cell activity factors.

26. The method of claim 24, wherein the sample is selected from the group consisting of whole blood, plasma, serum, urine, cerebrospinal fluid and lymphatic fluid.

27. A method of identifying an agent useful in the treatment of a mast cell activity-associated disorder or multiple sclerosis (MS), the method comprising steps of:
providing a candidate agent;
assessing the agent for a presence, level and/or location of an activity selected from the group consisting of stabilizing mast cell activity, inhibiting mast cell proliferation, inhibiting mast cell migration, inhibiting release of cytokines, inhibiting release of lipid derived metabolites, inhibiting release of granule-associated metabolites, enhancing hydration, reducing inflammation and combinations thereof.

28. The method of any one of claims 1-27, wherein the mast cell activity biomarker and/or the mast cell activity factor is selected from the group consisting of a cytokine, a preformed granule-associated metabolite, a lipid derived metabolite, chromogranin A, an immunoglobulin, a nucleic acid and combinations thereof.

29. The method of claim 28, wherein the cytokine is selected from the group consisting of IL-1, IL-2, IL-3, IL-4, IL-5, IL-6, IL-8, IL-17, IL-33, TNF-α, TGF-B, GM-CSF, MIP-1α, MIP-1β, INFγ, eosinophilic chemotactic factor and combinations thereof.

30. The method of claim 28, wherein the preformed granule-associated meditator is selected from the group consisting of histamine, N-methylhistamine, a proteoglycan, a neutral protease and combinations thereof.

31. The method of claim 30, wherein the proteoglycan is heparin and/or chondroitin sulphate.

32. The method of claim 30, wherein the neutral protease is selected from the group consisting of tryptase, chymase, carboxypeptidase, cathepsin G and combinations thereof.

33. The method of claim 28, wherein the lipid derived metabolite is a prostaglandin, a thromboxane and/or a leukotriene.

34. The method of claim 33, wherein the leukotriene is leukotriene E₄, leukotriene B₄ and/or leukotriene C₄.

35. The method of claim 33, wherein the prostaglandin is prostaglandin D₂, prostaglandin E₂, 11β-PGF_{2α} and/or tetranor-PGDM.

36. The method of claim 28, wherein the immunoglobulin is IgE.

37. The method of claim 28, wherein the nucleic acid is an RNA.

38. The method of claim 37, wherein the RNA is a messenger RNA.

39. The method of claim 38, wherein the RNA is determined using an expression array.

40. A kit for determining a MS susceptibility biomarker in a subject, the kit comprising:
agents for determining a presence, level and/or location of one or more particular mast cell activity factors, which one or more particular mast cell activity factors have been determined to contribute to a mast cell activity biomarker for MS susceptibility.
